(19) United States
(12) Patent Application Publication
(10) Pub. No.: US 2026/0069168 A1
(43) Pub. Date: Mar. 12, 2026

(54) WEARABLE COMPUTING DEVICE IDENTIFICATION AND USE FOR ROLE-BASED FEEDBACK

(71) Applicant: ZOLL Medical Corporation, Chelmsford, MA (US)

(72) Inventors: Binwei Weng, Andover, MA (US); Guy Robert Johnson, Wilton, NH (US); Gary Freeman, Waltham, MA (US)

(73) Assignee: ZOLL Medical Corporation, Chelmsford, MA (US)

(21) Appl. No.: 18/851,520

(22) PCT Filed: Mar. 22, 2023
(86) PCT No.: PCT/US2023/015955

§ 371 Date: Sep. 26, 2024

Related U.S. Application Data
(60) Provisional application No. 63/324,877, filed on Mar. 29, 2022.

(51) Int. Cl. A61B 5/11; A61B 5/00; G16H 40/67

(52) U.S. Cl. CPC A61B 5/1118; A61B 5/002; A61B 5/681; A61B 5/7203; G16H 40/67; A61B 2562/0219

(57) ABSTRACT

In an illustrative embodiment, a system for collecting and presenting metrics related to each member of a team of rescuers involved in a resuscitation effort includes a set of wearable devices, each configured to determine role-identifying data indicative of a role of a rescuer donning the respective device. The system may include a portable computing device for storing, during the resuscitation effort, a time progression of compression data and ventilation data, receiving the role-identifying data from the wearable devices, and storing a time series of role information representing a series of changes in roles among the team of rescuers. The system may include a remote computing system for preparing a case overview GUI presenting time periods of compression data, each time period being visually correlated with a current rescuer and/or current wearable device corresponding to the compression role.

### RELATED APPLICATIONS

This application is a U.S. National Phase application under 35 U.S. C. § 371 of International Patent Application No. PCT/US2023/015955 filed on Mar. 22, 2023, which claims priority to U.S. Provisional Patent Application Ser. No. 63/324,877, entitled “Wearable Computing Device Identification and Use for Role-Based Feedback,” filed Mar. 29, 2022. All above identified applications are hereby incorporated by reference in their entireties.

### BACKGROUND

Acute care is delivered to patients in emergency situations in the pre-hospital and hospital settings for patients experiencing a variety of acute medical conditions involving the timely diagnosis and treatment of disease states that, left alone, will likely degenerate into a life-threatening condition and, potentially, death within a period of 72 hours or less. Stroke, dyspnea (difficulty breathing), traumatic arrest, myocardial infarction and cardiac arrest are a few examples of disease states for which acute care is delivered to patients in an emergency setting. Acute care comprises different treatment and/or diagnosis, depending upon the disease state.

One example of acute care is cardio-pulmonary resuscitation (CPR), which is a process by which one or more acute care providers may attempt to resuscitate a patient who may have suffered a cardiac arrest or other acute adverse cardiac event by taking one or more actions, for example, providing chest compressions and ventilation to the patient. The first five to eight minutes of CPR, including chest compressions, are critically important, largely because chest compressions help maintain blood circulation through the body and in the heart itself. Ventilation is also key part of CPR because ventilations help to provide much needed gas exchange (e.g., oxygen supply and carbon dioxide deposit) for the circulating blood.

CPR may be performed by a team of one or more acute care providers, for example, an emergency medical services (EMS) team made up of emergency medical technicians (EMTs), a hospital team including medical caregivers (e.g., doctors, nurses, etc.), and/or bystanders responding to an emergency event. In some instances, one acute care provider can provide chest compressions to the patient while another can provide ventilations to the patient, where the chest compressions and ventilations may be time and/or coordinated according to an appropriate CPR protocol. When professionals such as EMTs provide care, ventilation may be provided via a ventilation bag that an acute care provider squeezes, for example, rather than by mouth-to-mouth. CPR can be performed in conjunction with electrical shocks to the patient provided by an external defibrillator, such as an automatic external defibrillator (AED). Such AEDs often provide instructions (e.g., in the form of audible feedback) to acute care providers, such as “Push Harder” (when the acute care provider is not performing chest compressions according to the desired depth), “Stop CPR,” “Stand Back” (because a shock is about to be delivered), and so on. In order to determine the quality of chest compressions being performed, certain defibrillators may obtain information from one or more accelerometers (such as those which are provided with e CPR D PADZ®, CPR STAT PADZ®, and ONE STEP™ pads made by ZOLL MEDICAL of Chelmsford, Mass.) that can be used to provide data to determine information such as depth of chest compressions (e.g., to determine that the compressions are too shallow or too deep and to thus cause an appropriate cue to be provided by the defibrillator).

### SUMMARY OF ILLUSTRATIVE EMBODIMENTS

The foregoing general description of the illustrative implementations and the following detailed description thereof are merely exemplary aspects of the teachings of this disclosure, and are not restrictive.

In one aspect, the present disclosure relates to a system for identifying role changes among a set of rescuers, the system including a set of wearable devices configured to be donned by a rescuer, each wearable device of the set of wearable devices including a display, processing circuitry, a wireless communication module, at least one sensor configured to detect motion, and a non-volatile storage medium, where the processing circuitry is configured via hardware logic and/or configured to execute software logic to perform a number of operations, the operations including monitoring a number of signals of the at least one sensor, collecting a set of motion data samples over a predetermined sample time period, using the set of motion data samples, calculating an activity value representing the predetermined sample time period, and transmitting, via the wireless communication module, the activity value. The system may include a computing device including processing circuitry, a wireless communication module, and a non-volatile storage medium, where the processing circuitry is configured via hardware logic and/or configured to execute software logic to perform a number of operations, the operations including receiving, from each wearable device of the set of wearable devices, a number of activity values, each activity value representing motions of the rescuer wearing the respective wearable device during a predetermined time period, storing, for each wearable device of the set of wearable devices in a storage region of the non-volatile storage medium, a time progression of activity values of the number of activity values received from the respective wearable device, and determining, in near real-time using the time progression of activity values for each device of the set of wearable devices, a given wearable device of the set of wearable devices worn by a rescuer actively performing chest compressions.

In some embodiments, the computing device is a portable computing device. The computing device may be a medical device.

In some embodiments, the processing circuitry of the set of wearable devices is configured to perform operations including detecting, from the number of signals, threshold motion of the respective wearable device, where each motion data sample of the set of motion data samples is collected based at least in part on the detecting. The number of sensor signals may represent at least one of acceleration of the wearable device, a gyroscopic progression of measurements sensed by the wearable device, or one or more magnetic field measurements sensed by the wearable device.

In some embodiments, the determining includes, for each wearable device of the set of wearable devices, calculating a combined activity value from the time progression of activity values corresponding to the respective wearable device, and comparing the combined activity values of each of the at least two wearable devices to identify a greatest combined activity value. Calculating the combined activity value may include calculating a root mean square (RMS) value. Calculating the combined activity value may include identifying a dominant frequency of compression rate. Calculating the combined activity value may include applying pattern matching to the time progression of values. Calculating the combined activity value may include calculating the combined activity value across multiple directions of motion. Determining the given wearable device most likely worn by the rescuer actively performing chest compressions may include storing, in a second non-volatile storage region, the combined activity value.

In some embodiments, the processing circuitry of the computing device is configured to perform operations including, prior to determining the given wearable device, applying noise suppression to the time progression of values. The wireless communication module of each wearable device of the set of wearable devices may be configured to communicate over a short-range communication protocol. The short-range communication protocol may be one of Bluetooth, Wi-Fi, radio frequency (RF), or Zigbee.

In some embodiments, the processing circuitry of the computing device is configured to perform operations including, responsive to determining the given wearable device is most likely worn by the rescuer actively performing chest compressions, provide compression data related to a respective depth of active compression to the processing circuitry of the given wearable device. The processing circuitry of the computing device may be configured to perform operations including formatting at least a portion of the compression data for presentation on the display of the given wearable device. Formatting the portion of the compression data may include presenting a current compression rate on the display of the given wearable device. Formatting the portion of the compression data may include presenting one of a set of color codes indicative of at least one of an acceptable compression rate or an unacceptable compression rate. Formatting the portion of the compression data may include presenting a current compression depth on the display of the wearable device. Formatting the portion of the compression data may include presenting one of a set of color codes indicative of at least one of an acceptable compression depth or an unacceptable compression depth. Formatting the portion of the compression data may include presenting an indication of sufficiency of release from compression depth. The processing circuitry of the computing device may be configured to perform operations including analyzing at least a portion of the compression data to prompt a timing of a next compression. Prompting the timing of the next compression may include providing at least one of an audible signal, a visual signal, or a haptic signal to the rescue via the given wearable device.

In some embodiments, the predetermined sample period is less than one second. The predetermined sample period may be between about a half a second and one second. Collecting the set of motion data samples may include storing each data sample of the set of motion data samples in a first-in-first-out (FIFO) queue. A number of the set of motion data samples may be at least three. The processing circuitry of each wearable device of the set of wearable devices may be configured to perform operations including, based on the number of sensor signals, detecting threshold motion of the wearable device, where the collecting is responsive at least in part to detecting the threshold motion. The processing circuitry of each wearable device of the set of wearable devices may be configured to perform operations including obtaining, from the computing device, indication of active chest compressions, where the determining is responsive at least in part to obtaining the indication of the active chest compressions.

In some embodiments, determining the given wearable device most likely worn by the rescuer actively performing chest compressions includes comparing a timestamp associated with each activity value of the time progression of activity values with a period of time of active chest compressions. Storing the time progression of activity values may include storing each value of the time progression of activity values with a corresponding timestamp.

In some embodiments, the processing circuitry of the computing device is configured to perform operations including setting a role of the rescuer wearing the given wearable device to a compression delivery role, where a set of roles includes the compression delivery role and a non-compression delivery role. Setting the role of the rescuer wearing the given wearable device may include providing, to a patient monitoring device, an identifier associated with the given wearable device. Setting the role of the rescuer may include switching the role of the rescuer from a prior wearable device of the set of wearable devices to the given wearable device. Setting the role of the rescuer may include beginning a new epoch of a time series of epochs, each epoch corresponding to compression delivery by a same rescuer of the rescuers. Each epoch of the time series of epochs may include at least one compression interval, where each compression interval includes a compression delivery time period and a ventilation time period.

In some embodiments, determining the given wearable device most likely worn by the rescuer actively performing chest compressions includes confirming that each time progression of activity values includes at least a threshold number of values. The threshold number of values may be at least three. Storing the time progression of activity values may include storing up to the threshold number of values.

In one aspect, the present disclosure relates to a method for identifying role changes among a set of rescuers, the method including monitoring, by processing circuitry of a wearable device configured to be donned by a rescuer, a number of sensor signals representing motion of the wearable device, collecting, by the processing circuitry, a set of motion data samples over a predetermined sample time period, using the set of motion data samples, calculating, by the processing circuitry, an activity value representing the predetermined sample time period, and providing, via a wireless communication module of the wearable device, the activity value to a separate computing device. Processing circuitry of the separate computing device may be configured to determine, in near real-time, based on a number of activity values collected from a number of wearable devices including the wearable device, a given wearable device reporting a respective activity value most likely indicative of chest compression delivery.

In some embodiments, the wearable device is configured to be mounted to the wrist of the rescuer. The separate computing device may be a portable computing device. The separate computing device may be a medical device. An accelerometer of the wearable device may generate the number of sensor signals. The set of motion data samples may be a set of acceleration data samples.

In some embodiments, determining the given wearable device includes calculating, from the number of activity values for each wearable device of the number of wearable devices, a combined activity value. Calculating the combined activity value may include calculating a root mean square (RMS) value. Calculating the combined activity value may include identifying a dominant frequency of compression rate. Calculating the combined activity value may include applying pattern matching to the time progression of values.

In some embodiments, the method includes receiving, from the separate computing device, indication of active compression delivery, where the collecting, the calculating, and the providing are executed responsive in part to the active compression delivery. The method may include, prior to calculating the activity value, applying, by the processing circuitry, noise suppression to the set of motion data samples.

In some embodiments, the wireless communication module is configured to communicate over a short-range communication protocol. The short-range communication protocol may be one of Bluetooth, Wi-Fi, radio frequency (RF), or Zigbee.

In some embodiments, the processing circuitry of the separate computing device is configured to, responsive to determining the wearable device is the given wearable device, provide compression data related to a respective depth of active compression to the processing circuitry of the wearable device. The method may include formatting at least a portion of the compression data for presentation on a display of the wearable device. Formatting the portion of the compression data may include presenting a current compression rate on the display of the wearable device. Formatting the portion of the compression data may include presenting one of a set of color codes indicative of at least one of an acceptable compression rate or an unacceptable compression rate. Formatting the portion of the compression data may include presenting a current compression depth on the display of the wearable device. Formatting the portion of the compression data may include presenting one of a set of color codes indicative of at least one of an acceptable compression depth or an unacceptable compression depth. Formatting the portion of the compression data may include presenting an indication of sufficiency of release from compression depth. The method may include analyzing at least a portion of the compression data to prompt a timing of a next compression. Prompting the timing of the next compression may include providing at least one of an audible signal, a visual signal, or a haptic signal to the rescue via the wearable device.

In some embodiments, the predetermined sample time period is less than one second. The predetermined sample time period may be between about a half a second and one second. Collecting the set of motion data samples may include storing each data sample of the set of motion data samples in a first-in-first-out (FIFO) queue. A number of the set of motion data samples may be at least three. Calculating the activity value may include calculating the activity value across multiple directions of motion. Providing the activity value may include providing the activity value along with a unique identifier assigned to the wearable device. The method may include, based on the number of sensor signals, detecting, by the processing circuitry, threshold motion of the wearable device, where the collecting is responsive at least in part to detecting the threshold motion.

In one aspect, the present disclosure relates to a method for identifying role changes among a set of rescuers, the method including receiving, by processing circuitry of a computing device from each wearable device of at least two wearable devices worn by at least two rescuers at an emergency medical scene, a number of values, each value representing motion of the respective wearable device during a predetermined time period, storing, for each wearable device of the at least two wearable devices in a non-volatile storage region, a time progression of values of the number of values received from the respective wearable device, determining, by the processing circuitry in near real-time, a given wearable device of the at least two wearable devices most likely worn by the rescuer of the at least two rescuers actively performing chest compressions, where the determining includes for each wearable device of the at least two wearable devices, calculating an activity value from the time progression of values corresponding to the respective wearable device, and comparing the activity values of each of the at least two wearable devices to identify a greatest activity value, and setting, by the processing circuitry, a role of the rescuer wearing the wearable device corresponding to the greatest activity value, to a compression delivery role, where a set of roles includes the compression delivery role and a non-compression delivery role.

In some embodiments, the method includes obtaining, by the processing circuitry from a chest compression monitoring device, indication of active chest compressions, where the determining is responsive at least in part to obtaining the indication of the active chest compressions. Determining the given wearable device most likely worn by the rescuer actively performing chest compressions may include comparing a timestamp associated with each value of the time progression of values with a period of time of active chest compressions. Storing the time progression of values may include storing each value of the time progression of values with a corresponding timestamp.

In some embodiments, setting the role of the rescuer wearing the wearable device corresponding to the greatest activity value includes providing, to a patient monitoring device, an identifier associated with the wearable device corresponding to the greatest activity value or the rescuer wearing the wearable device. Determining the given wearable device most likely worn by the rescuer actively performing chest compressions may include confirming that the time progression of values corresponding to each wearable device of the at least two wearable devices includes at least a threshold number of values. The threshold number of values may be at least three. Storing the time progression of values may include storing up to the threshold number of values.

In some embodiments, determining the given wearable device most likely worn by the rescuer actively performing chest compressions includes storing, in a second non-volatile storage region, the activity value. The method may include supplying compression data for display at the given wearable device. The compression data may include a compression depth. The compression data may be obtained by the processing circuitry via a network from a chest compression monitoring device. The network may be a wireless network.

In some embodiments, setting the role of the rescuer includes switching the role of the rescuer from a prior wearable device of the at least two wearable devices to the given wearable device. Setting the role of the rescuer may include beginning a new epoch of a time series of epochs, each epoch corresponding to compression delivery by a same rescuer of the rescuers. Each epoch of the time series of epochs may include at least one compression interval, where each compression interval includes a compression delivery time period and a ventilation time period.

In one aspect, the present disclosure relates to a system for distributing and coordinating resuscitation information among a team of rescuers, the system including a medical device including processing circuitry, and a wireless communication module, where the processing circuitry is configured via hardware logic and/or configured to execute software logic to perform a number of operations, the operations including receiving sensor data from one or more sensors, analyzing the sensor data to determine a compression depth of a chest of a patient, and transmitting, via the wireless communication module, compression data including the compression depth. The system may include a portable computing device including processing circuitry, and at least one wireless communication module, where the processing circuitry is configured via hardware logic and/or configured to execute software logic to perform a number of operations, the operations including receiving, via the at least one wireless communication module, the compression data from the medical device, formatting the compression data for presentation by a wearable device, and transmitting, via the at least one wireless communication module, the formatted compression data for use by the wearable device. The wearable device may be configured to be donned by a rescuer, the wearable device including a display, processing circuitry, and a wireless communication module, where the processing circuitry is configured via hardware logic and/or configured to execute software logic to perform a number of operations, the operations including receiving, via the at least one wireless communication module, the formatted compression data, and presenting, on the display, the formatted compression data, where the formatted compression data is configured to provide the rescuer in an indication of quality of the compression depth.

In some embodiments, the portable computing device includes a non-volatile storage medium, and the processing circuitry of the portable computing device is configured to perform the operations including storing, to the non-volatile storage medium, an identifier of the wearable device. The processing circuitry of the portable computing device may be configured to perform the operations including storing a second identifier of a second wearable device, formatting the compression data as second formatted compression data for presentation by the second wearable device, and transmitting, based in part on the second identifier for use by the second wearable device, the second formatted compression data. The wearable device may be donned by the rescuer supplying compressions to a patient, and the second wearable device may be donned by a different user. The different user may be a supervisor of the rescuer or a medical professional.

In some embodiments, the medical device includes a ventilator. The medical device may be a portable chest compression monitoring device. The indication of the quality of the compression depth may include a color indicator representing one of insufficient depth, sufficient depth, or over-compressed depth. The indication of the quality may include one of an audible indication or a tactile indication.

In some embodiments, the wearable device is one of a set of wearable devices, and transmitting the formatted compression data for use by the wearable device includes identifying, from the set of wearable devices, a given wearable device donned by the rescuer supplying compressions to a patient. The portable computing device may include a non-volatile storage medium, and the processing circuitry of the portable computing device may be configured to perform the operations including storing the compression data to the non-volatile storage medium, repeating the receiving, formatting, transmitting, and storing over time, and analyzing a time period of the stored compression data to determine one or more compression metrics. The portable computing device may be a tablet style portable computer.

In some embodiments, the portable computing device includes a display, and the processing circuitry of the portable computing device is configured to perform the operations including presenting, on the display, the compression data. The processing circuitry of the portable computing device may be configured to perform the operations including formatting the compression data as second formatted compression data, and presenting, on the display of the portable computing device, the compression data may include presenting the second formatted compression data. Presenting the second formatted compression data may include presenting an identification of at least one of the wearable device or the rescuer.

In some embodiments, the computing device is another medical device. The wearable device may be a smart watch. The system may include a second wearable device, where the processing circuitry of the portable computing device may be configured to perform the operations including receiving, via the at least one wireless communication module, ventilation data, formatting the ventilation data for presentation by the second wearable device, and transmitting, via the at least one wireless communication module, the formatted ventilation data for use by the second wearable device. Transmitting the formatted ventilation data may include transmitting the formatted ventilation data for use by both the wearable device and the second wearable device. Receiving the ventilation data may include receiving the ventilation data from the medical device. The second wearable device may include a display, processing circuitry, and a wireless communication module, where the processing circuitry may be configured via hardware logic and/or configured to execute software logic to perform a number of operations, the operations including receiving, via the at least one wireless communication module, the formatted ventilation data, and presenting, on the display, the formatted ventilation data, where the formatted ventilation data may be configured to provide a wearer of the second wearable device with an indication of quality of a ventilation volume. The indication of quality of the ventilation volume may include a color indicator representing one of insufficient volume, sufficient volume, or excessive volume. The indication of quality of the ventilation volume may include one of an audible indication or a tactile indication.

In one aspect, the present disclosure relates to a system for collecting and presenting metrics related to each member of a team of rescuers involved in a resuscitation effort, the system including a set of wearable devices, each wearable device of the set of wearable devices including processing circuitry, where the processing circuitry is configured via hardware logic and/or configured to execute software logic to perform a number of operations, the operations including determining role-identifying data indicative of a role of a rescuer donning the respective wearable device, where the role is one of a set of roles including a compression role and a non-compression role, and transmitting, via the wireless communication module, the role-identifying data. The system may include a portable computing device including processing circuitry, at least one wireless communication module, and a non-volatile storage medium, where the processing circuitry is configured via hardware logic and/or configured to execute software logic to perform a number of operations, the operations including storing, during a resuscitation effort by a team of rescuers wearing the set of wearable devices, a time progression of compression data and ventilation data, receiving, during the resuscitation effort from at least one wearable device of the set of wearable devices, the respective role-identifying data, where one or more wearable devices transmit role-identifying data at times throughout the resuscitation effort, thereby indicating a change in role of the corresponding rescuer, during the resuscitation effort and responsive to the change in role, correlating the compression data with a current rescuer of the team of rescuers and/or a current wearable device of the set of wearable devices corresponding to the compression role, and formatting, for presentation to a user at a display device, the time progression of the compression data, where formatting includes visually identifying, for each time period of a number of time periods, the current rescuer corresponding to the compression role.

In some embodiments, the non-compression role includes a ventilation role. The operations performed by the processing circuitry of the portable computing device may include, during the resuscitation effort and responsive to the change in role, correlating ventilation data with a given rescuer of the team of rescuers and/or a given wearable device of the set of wearable devices corresponding to the ventilation role. The processing circuitry of the portable computing device may be configured to perform the operations including formatting, for presentation to a user at a display device, the time progression of the ventilation data, where formatting includes visually identifying, for each time period of the number of time periods, the current rescuer corresponding to the ventilation role.

In some embodiments, determining the role-identifying data includes receiving, via a user interface of the respective wearable device, selection of a current role of the set of roles. The number of time periods may include a number of epochs, a beginning of each new epoch corresponding to the change in the role among the team of rescuers. Formatting the time progression of the compression data may include formatting the time progression of the compression data in real-time or near-real-time. Formatting the time progression of the compression data may include formatting for presentation to the user at a display of the portable computing device.

In some embodiments, the processing circuitry of the portable computing device is configured to perform the operations including determining, based at least in part on the respective role-identifying data, the role of another wearable device of the set of wearable devices. Formatting the time progression of the compression data may include color-coding the compression data, for each period of the number of time periods, as one of insufficient compression, sufficient compression, or over-compression. Formatting the time progression of the compression data may include color-coding the compression data, for compression of a number of chest compressions, as one of insufficient compression, sufficient compression, or over-compression.

In some embodiments, formatting the time progression of the compression data includes determining, for each period of the number of time periods, a compression rate. Formatting the time progression of the compression data may include color-coding, for each period of the number of time periods, the compression rate as slow, sufficient, or fast.

In some embodiments, the processing circuitry of the portable computing device is configured to perform the operations including determining, for each period of the number of time periods, sufficiency of at least one of compression depth or compression rate, identifying the compression depth and/or compression rate represents significantly decreased performance by a corresponding rescuer, and responsive to identifying the significantly decreased performance, issuing a recommendation of role-switching among the team of rescuers. Issuing the recommendation may include presenting, at a display of the portable computing device, the recommendation. Issuing the recommendation may include transmitting the recommendation to a corresponding wearable device of the set of wearable devices for presentation by the corresponding wearable device.

In one aspect, the present disclosure relates to a system for collecting and presenting metrics related to each member of a team of rescuers involved in a resuscitation effort, the system including a set of wearable devices, each wearable device of the set of wearable devices including processing circuitry, where the processing circuitry is configured via hardware logic and/or configured to execute software logic to perform a number of operations, the operations including determining role-identifying data indicative of a role of a rescuer donning the respective wearable device, where the role is one of a set of roles including a compression role and a non-compression role, and transmitting, via the wireless communication module, the role-identifying data. The system may include a portable computing device including processing circuitry, at least one wireless communication module, and a non-volatile storage medium, where the processing circuitry is configured via hardware logic and/or configured to execute software logic to perform a number of operations, the operations including storing, during a resuscitation effort by a team of rescuers wearing the set of wearable devices, a time progression of compression data and ventilation data, receiving, during the resuscitation effort from at least one wearable device of the set of wearable devices, the respective role-identifying data, where one or more wearable devices transmit role-identifying data at times throughout the resuscitation effort, and storing, during the resuscitation effort responsive at least in part to receiving the respective role-identifying data, a time series of role information including at least one of i) the role-identifying data or ii) an identification of a rescuer or a wearable device corresponding to a compression role of a set of roles including the compression role and a non-compression role, where the time series of role information represents a series of changes in roles among the team of rescuers. The system may include a remote computing system including processing circuitry configured via hardware logic and/or configured to execute software logic to perform a number of operations, the operations including preparing, for review by a user at a display, a case overview graphical presentation where preparing includes, for each change of role in the series of changes in roles, correlating the time progression of compression data with a current rescuer of the team of rescuers and/or a current wearable device of the set of wearable devices corresponding to the compression role, and formatting, for presentation to a user at a display device, the time progression of the compression data, where formatting includes visually identifying, for each time period of a number of time periods, the current rescuer corresponding to the compression role.

In some embodiments, the non-compression role includes a ventilation role. Preparing the case overview graphical presentation may include correlating ventilation data with a given rescuer of the team of rescuers and/or a given wearable device of the set of wearable devices corresponding to the ventilation role.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1A through FIG. 1C illustrate example medical emergency scenes including rescuers donning wearable computing devices configured for role-based feedback;

FIG. 2 illustrates an example environment for analyzing data collected from wearable computing devices donned by rescuers performing various roles at a medical emergency scene;

FIG. 3A through FIG. 3M illustrate example display presentations for a wearable computing device configured for role-based feedback;

FIG. 4A through FIG. 4E illustrate example screen shots of medical emergency scene metrics derived in part through analyzing data collected from wearable computing devices donned by rescuers at a medical emergency scene;

FIG. 5A through FIG. 5D illustrate example screen shots of a portable computing device configured for role-based feedback to a supervisor overseeing rescuers donning wearable computing devices at an medical emergency scene;

FIG. 6A is a flow chart of an example method for calculating an activity value corresponding to motions of a wearable computing device;

FIG. 6B-1 and FIG. 6B-2 illustrate a flow chart of an example method for applying activity values corresponding to motions of a set of wearable computing devices to determine which wearable computing device is donned by a rescuer performing chest compressions;

FIG. 7A and FIG. 7B illustrate a flow chart of an example method for tracking role changes of a team of rescuers throughout a rescue at an emergency medical scene;

FIG. 8A through 8D illustrate a flow chart of an example method for identifying roles of each rescuer in a team of rescuers at an emergency medical scene;

FIG. 9A is a screen shot of an example performance report presenting chest compression-related metrics corresponding to rescuers at an emergency medical scene;

FIG. 9B is a screen shot of an example performance report presenting ventilation-related metrics corresponding to rescuers at an emergency medical scene; and

FIG. 10 is a block diagram of an example system of computing devices configured for use at an emergency medical scene.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment. CPR guidelines may be provided to identify target performance metrics for the CPR provider (e.g., wearer of the watch or other wearable feedback device). Feedback devices of embodiments of the present disclosure may be programmed to provide user feedback based on such target performance metrics, such as for example, a target range of rate of compression (e.g., number of chest compressions per minute), a target range of depth of compression (e.g., downward displacement distance of the thorax), a target range of ventilation tidal volume (e.g., volume of an inspiratory positive pressure breath delivered to the patient), and a target range of ventilation rate (e.g., number of breaths per minute, number of breaths between cycles of N compressions, such as 2 breaths per 30 compressions or 2 breaths per 30 compressions, designed to provide lifesaving support to an individual without causing undue harm. These guidelines may be updated from time-to-time. Further, guidelines vary depending upon whether the victim is an adult, a child, or an infant. to maximize effectiveness. For example, according to the 2020 American Heart Association (AHA) guidelines, the target adult compression depth is within a range of 2.0-2.4 inches (5-6 cm), while infant compression varies (e.g., about one-third of the anterior-posterior diameter) being typically between approximately 4 cm (1.5 inches) for younger pediatric victims and lapproximately 5 cm (2 inches) for older pediatric victims. Target ventilation tidal volume is commonly based on an ideal body weight (IBW) calculated using the gender and height of the victim. For example, target ventilation volume may be calculated as between 6 to 8 mL per kilogram IBW. Target ventilation rate is typically within a range of 8 to 10 breaths per minute. Target performance metrics may be appropriately tailored according to updated guidelines.

When performing CPR on a patient at an emergency scene, the various rescue personnel may begin with one rescuer performing each task (e.g., a first rescuer performing chest compressions while a second rescuer provides ventilation). In some embodiments, a rescuer may receive feedback from a hand-mounted device or watch style computing device to guide the rescuer in reaching and maintaining efforts within CPR guidelines. For example, U.S. Pat. No. 10,092,236 entitled “Emergency Medical Services Smart Watch” and issued Oct. 9, 2018, incorporated by reference herein in its entirety, describes a wrist-worn device configured to provide CPR feedback to a rescuer. In another example, U.S. Pat. No. 10,912,709 entitled “Hand Mounted CPR Chest Compression Monitor” and issued Feb. 9, 2021, incorporated by reference herein in its entirety, describes a monitor for measuring depth of chest compressions during CPR and displaying feedback regarding provision of chest compressions. U.S. Pat. No. 11,202,579 entitled “Wrist-Worn Device for Coordinating Patient Care” and issued Dec. 21, 2021, incorporated by reference herein in its entirety, describes a wrist-mounted device configured to provide feedback to a rescuer related to performance of resuscitation activities as well as to a supervisor monitoring the rescue operation. For example, based upon compression rate and/or compression depth as determined by the wrist-mounted device being outside of recommended guidelines, the supervisor may decide to switch the rescuer performing CPR to another task, allowing that rescuer to rest. In accordance with aspects of the present disclosure, it would be advantageous for the system to have algorithms in place that facilitate quick and automatic identification of actions particular rescuers are performing, for example, whether it be chest compressions, ventilations or another type of action. This allows for a more streamlined workflow whether during an emergency rescue situation and/or for post-case review for quality care, evaluation, and training purposes.

In one aspect, the present disclosure relates to a system that is configured to identify role changes among a set of rescuers each donning a wearable device configured to provide feedback related to performance of resuscitation activities. Each wearable device may be configured to monitor sensor signals to determine motion data related to movements of the rescuer. Motion data collected over time may be used to calculate activity values representative of movements of the wearer. The activity values of each of the rescuers may be analyzed individually and/or in comparison to identify an activity type more likely being performed by at least a portion of the rescuers. Using the identified activity types, a corresponding role may be assigned or confirmed for at least some of the rescuers, such as a rescuer performing chest compressions and/or a rescuer providing ventilation.

In some implementations, once the particular activity type or rescuer role is identified, the role identifications are used to supply appropriate feedback related to the activities of each individual rescuer. For example, a rescuer identified as providing chest compressions may be provided with coaching to maintain compression rate and/or depth within CPR guidelines, while a rescuer identified as providing ventilation may be provided with coaching for ventilation timing and volume. If the rescuers switch roles, the automatic role change identification can modify the feedback directly on the particular device worn and/or otherwise associated with that rescuer, so that the information provided at each wearable device is both appropriate to the present role of the rescuer and simplified to present only the information appropriate to that role. Thus, the user interface and/or other feedback (e.g., audible, haptic, etc.) may be streamlined and easy to follow. Such streamlined and simplified workflow can be particularly important and advantageous in an emergency situation, so as to reduce unnecessary distractions and help keep the rescuer focused and on task.

The role identifications, in some implementations, are used to collect performance data related to each individual rescuer's performance related to one or more rescue activities. The performance data, for example, may be used during and/or after rescue activities to provide a supervisor or other individual with analytics for discerning relative performance among team members. During rescue activities, for example, the supervisor may recognize diminished performance in the rescuer performing chest compressions which takes strength, concentration, and endurance. To provide that rescuer with support, the supervisor may request that rescuers at the scene switch roles, or the supervisor may at least check in with that particular rescuer to see what can be done to improve the overall performance of treatment, e.g., for compressions or ventilations. In another example, after rescue activities, performance data may be analyzed to compare rescuer to rescuer, since the performance metrics (e.g., rate, depth, volume, timing, etc.) may be tracked in relation to each individual rescuer through the automatic role identification. In this manner, in an illustrative example, rescuers may be identified for further training based on lower performance metrics among their peers.

In one aspect, the present disclosure relates to a wearable computing device configured to collect motion data and provide activity value calculations usable in identifying the type of activity that particular rescuers are performing, which corresponds to roles of individual rescuers involved in emergency resuscitation activities. The wearable computing device, for example, may include at least one sensor generating signals representing motion of the wearable device. The wearable computing device may include a wireless communication module for providing activity values, calculated by the wearable computing device based on the sensor signals, to a separate computing device for determining the role of the rescuer wearing the wearable computing device. In this manner, rather than needing to remember to manually switch roles when busy at a rescue scene, the wearable computing device may provide information to a separate computing device, such as a medical device (e.g., defibrillator, defibrillator/monitor, patient monitor) or portable computing device (e.g., tablet, smart phone, etc.) for analysis and comparison to activity values supplied by other wearable computing devices at the emergency medical scene.

In some implementations, the wearable computing device is configured to receive feedback from the separate computing device based on the determined role. For example, the rescuer performing the role of compression delivery may receive prompting related to cessation of compressions, periodically, for ventilation of the patient.

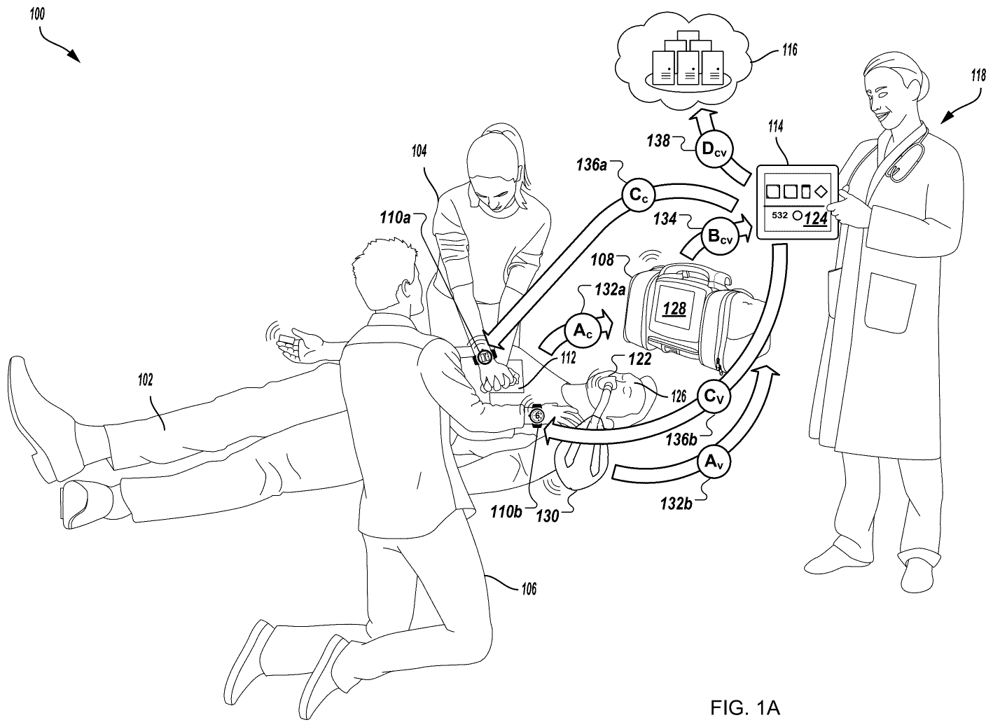

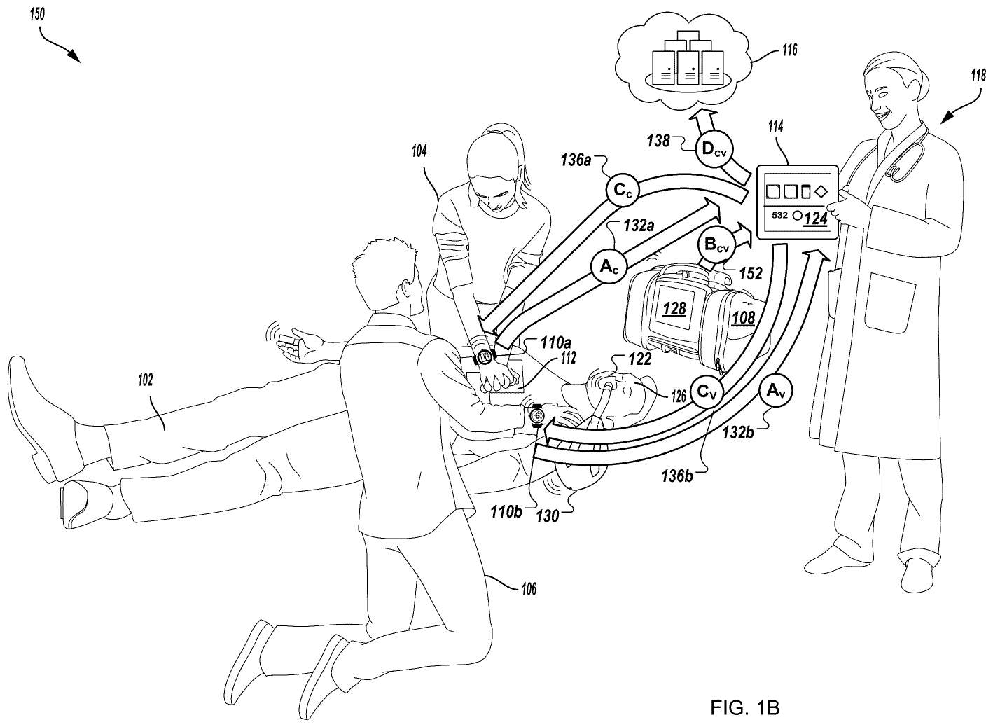

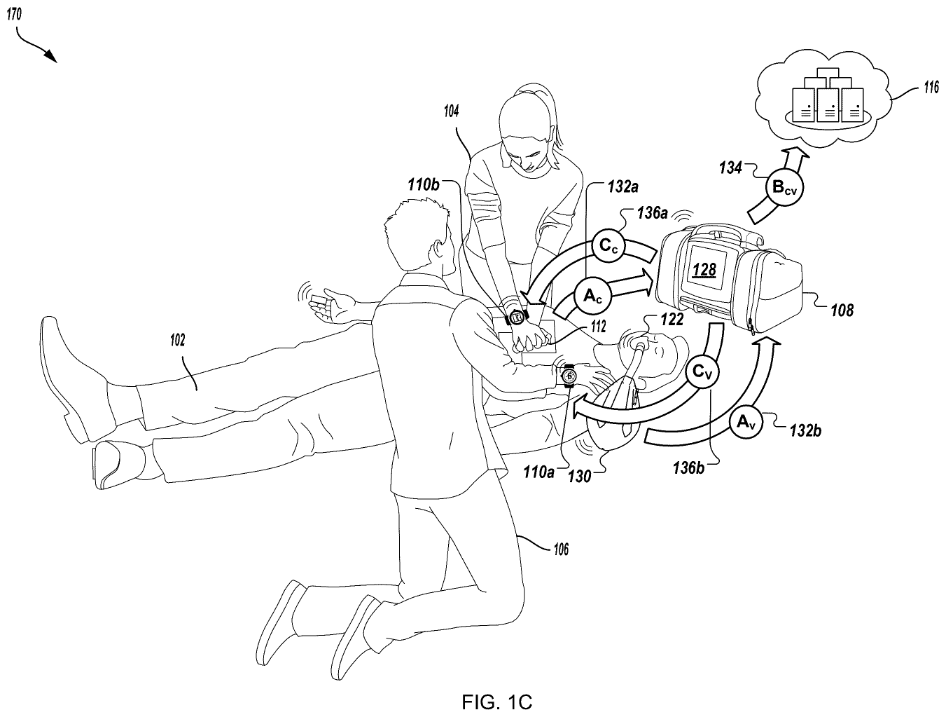

FIG. 1A through FIG. 1C illustrate example medical emergency scenes including rescuers donning wearable computing devices configured for role-based feedback. Referring to FIG. 1A, at an emergency care scene 100, a rescuer 104 performs cardiopulmonary resuscitation (CPR) on a victim or patient 102 (the terms are used interchangeably herein to indicate a person who is the subject of intended or actual CPR and related treatment, or other medical treatment), such as an individual who has apparently undergone sudden cardiac arrest. The emergency care scene 100 can be, for instance, at the scene of an accident or health emergency, in an ambulance, in an emergency room or hospital, or another type of emergency situation. The rescuer 104 can be, for instance, a civilian responder with limited or no training in lifesaving techniques; a first responder, such as an emergency medical technician (EMT), police officer, or firefighter; or a medical professional, such as a physician or nurse. The rescuer 104 may be acting alone or may be acting with assistance from one or more other rescuers, such as an assisting rescuer 106 (e.g., a partner EMT). In the example of FIG. 1A, the rescuer 104 is shown delivering chest compressions to the patient 102 and the rescuer 106 is shown deliver ventilations to the patient using a ventilation device 112 (e.g., bag-valve mask).

In the illustration of scene 100, the rescuers 104, 106 are shown having deployed a defibrillator/monitor 108, such as an automated external defibrillator (AED), a professional defibrillator/monitor, patient monitor, or another type of defibrillating and/or monitoring apparatus, to treat the patient 102. The defibrillator/monitor 108 is connected to electrode pads 112 intended to be placed on the patient's chest via one or more cables. The electrode pads 112 may acquire signals indicative of the patient's ECG. The defibrillator/monitor 108 may analyze those signals and, if the signals determine that the patient is in need of defibrillation, provide defibrillation treatment to the patient 102 as appropriate through the electrode pads 112.

The rescuers 104, 106, in some embodiments, use wearable devices 110a, 110b, respectively, such as a smart watch or wrist-mounted computer as illustrated, to assist in treating the patient 102. In other examples, the wearable devices 110 may include smart glasses or a protective visor with augmented reality display. To assist in resuscitation activities, for example, the wearable devices 110a, 110b can provide prompting to the rescuers 104, 106 to assist the rescuers 104, 106 in delivering chest compressions, ventilations, mouth-to-mouth resuscitation, defibrillation, or other treatments to the patient 102.

In some implementations, a supervisor 118 uses a portable computing device 114, such as a tablet computer or notebook computer, to coordinate treatment provided by the multiple rescuers 104, 106. The portable computing device 114 may be in communication with the wearable devices 110a, 110b and/or the defibrillator/monitor 108. Additional computing devices, such as laptop computers or computing devices integrated into an ambulance, can be used to analyze health data about the patient or data indicative of treatment delivered to the patient or to communicate such data to a remote location (e.g., a dispatch center, an emergency room, or a remote server).

One or more sensors (e.g., sensors 122, 126) can be used to monitor the patient 102. For instance, the sensors 122, 126 may monitor parameters indicative of the patient's health status, e.g., physical parameters such as the patient's heart rate, electrocardiogram (ECG), blood pressure, temperature, respiration rate, blood oxygen level, end-tidal carbon dioxide level (ETCO2), pulmonary function, blood glucose level, or other parameters indicative of the patient's health status. Some sensors, such as heart rate or ECG sensors, can be included in pads 112 of the defibrillator/monitor 108. Further, in some embodiments, one or more sensors in the pads 112 may collect signals indicative of rate and/or depth of compressions. One or more sensors may monitor the treatment delivered to the patient 102. For instance, a compression puck can be positioned beneath the hands of rescuer 104 as the rescuer 104 administers CPR to detect a rate, depth, and/or duration of compressions delivered to the patient 102. Additionally, an airflow sensor 122 on the ventilation device 130 can monitor volume and rate of ventilations administered to the patient 102 by rescuer 106. Some sensors can monitor both parameters indicative of the patient's health status and parameters indicative of the treatment delivered to the patient. For example, ventilation sensors 122 can provide information about the patient's health status or information about the treatment.

In some implementations, the wearable devices 110a, 110b are configured to collect activity values related to movements of the rescuers 104, 106. The activity values, for example, may be calculated based on sensor signals collected by one or more sensors of each wearable device 110a, 110b. The activity values may each be associated with a timestamp corresponding to the activity sensed by the wearable device 110a or the wearable device 110b. The wearable devices 110a, 110b, in some embodiments, provide activity values 132a (e.g., compression related motion data A_{C}) and activity values 132b (e.g., ventilation related motion data A_{V}) to the defibrillator/monitor 108. The activity values 132a, 132b, for example, may be provided to the defibrillator/monitor 108 via a wireless communications interface with the defibrillator/monitor 108.

In some implementations, the defibrillator/monitor 108 performs calculations on the activity values supplied by the wearable devices 110a, 110b to determine which wearable device 110a, 110b is most likely worn by the rescuer 104, 106 performing chest compressions. The defibrillator/monitor 108, for example, may compare activity values and/or a combined metric calculated using the activity values corresponding to the first wearable device 110a to activity values and/or a combined metric calculated using the activity values corresponding to the second wearable device 110b. In another example, the defibrillator/monitor 108 may compare the activity values and/or a combined metric calculated using the activity values corresponding to each wearable device 110a, 110b to a compression activity profile. The compression activity profile, in some examples, may be in the form of a range of values, a pattern of values (or pattern of ranges), or at least one threshold value. In a further example, the defibrillator/monitor 108 may compare the activity values and timing of each activity value with timing of performance of chest compressions (e.g., as determined via sensors in the pads 112 or upon another device).

The defibrillator/monitor 108, in some implementations, presents an identification of at least one rescuer 104, 106 and the detected role of the rescuer 104, 106 in a display region 128 of the defibrillator/monitor 108. For example, the defibrillator/monitor 108 may present compression depth and/or rate information along with an indication of the rescuer 104 performing the chest compressions. The indication or identification of the rescuer 104, in some examples, may include a wearable device identification code, an employee identifier associated with the wearable device 110a, 110b (e.g., as looked up based on a unique identifier provided in the data 132a, 132b), or at least part of a name of the rescuer 104 (e.g., full name, first initial and last name, last name only, initials only, etc.).

The defibrillator/monitor 108, in some implementations, is configured to combine the activity values 132a, 132b and/or the indications of roles of the rescuers 104a, 104b with other data collected by the defibrillator/monitor 108, and forward compression, ventilation, and defibrillator/monitor data B_{CV} 134 to the portable device 114 of the supervisor 118. The defibrillator/monitor data portion of the data B_{CV} 134, for example, can include patient data including one or more types of data discussed above. Further, the defibrillator/monitor data can include indications of compression timing and/or rate (e.g., from sensor data collected by the pads 112 of the defibrillator/monitor 108 or a separate monitoring device such as a compression puck) as well as ventilation timing and/or volume (e.g., collected by the defibrillator/monitor 108 from the airflow sensor 122 of the ventilation device 130).

In some implementations, the portable device 114 receives the data B_{CV} 134 and generates, based on the data, a supervisor user interface for presentation in a display region 124 of the portable device 114. The supervisor user interface, for example, may include performance metrics related to both the chest compressions performed by the first rescuer 104 and the ventilations provided by the second rescuer 106. In some embodiments, the user interface replicates or presents similar information as the information presented in the display region 128 of the defibrillator/monitor 108.

The portable device 114, in some implementations, provides compression coaching or prompting data C_{C} 136a to the wearable device 110a worn by the rescuer 104 performing chest compressions. The data C_{C} 136a, for example, may include real-time depth information to assist in guiding the rescuer 104 in adjusting compressions to maintain depth within the recommended range for the patient 102. In another example, the data C_{C} 136a may include real-time rate information to support the rescuer 104 in maintaining a compression rate within the recommended range for the patient 102. Further, the data C_{C} 136a may include information useful in prompting the rescuer 104 to pause from performing compressions to allow for ventilations to be delivered.

In some implementations, the portable device 114 provides ventilation coaching or prompting data C_{V} 136a to the wearable device 110b of the rescuer 106 performing ventilations for the patient 126. The data C_{V} 136a, for example, may include real-time assistance in ventilation timing and/or ventilation volume.

Although described as being separate types of data 136a, 136b, in some embodiments, each wearable device 110a, 110b receives a same set of data along with an indication of role corresponding to that particular device 110a, 110b. Using the indication of role, for example, the wearable device 110a, 110b can generate coaching or prompting output appropriate to the indicated role.

In some implementations, the portable device 114 provides compression and ventilation data D_{CV} 138 to a remote computing system 116. The data D_{CV} 138, for example, can include any of the information provided in the data B_{CV} 134, transferred from the defibrillator/monitor 108 to the portable device 114. The remote computing system 116, in some examples, may include one or more servers such as a cloud server hosting data storage and/or web portals for a medical treatment system. The remote computing system 116, in one example, may collect historical data over time for each of the rescuers 104, 106 to assist in performance tracking. In another example, the remote computing system 116 may receive the data D_{CV} 138 in real-time or near real-time, allowing a remotely located medical professional to review interventions provided at the emergency scene 100. The ability to transfer data to the remote computing system 116 in real-time or near real-time may be beneficial, for example, where no supervisor 118 is available and/or where lay persons are performing CPR on the patient 102. In some embodiments, the data B_{CV} transferred to the portable device 114 from the defibrillator/monitor 108 may be transferred directly to the remote computing system 116 without needed to transmit through the portable device 114.

Although the motion data 132a, 132b is illustrated as being provided to the defibrillator/monitor 108, in other implementations, the wearable devices 110a, 110b are only configured to communicate with the portable computing device 114. Additionally, in further implementations, one or more other wearable devices 110 may be deployed to the emergency scene 100, such as a wearable device 110 worn by the supervisor 118. For example, the supervisor 118 may at some point rotate into performing chest compressions or ventilations, while one of the rescuers 104, 106 monitors progress via the portable computing device 114.

Turning to FIG. 1B, in an example emergency scene 150, the rescuers 104, 106 are wearing the wearable devices 110a, 110b and performing chest compressions and ventilations on the patient 102. As in FIG. 1A, the wearable devices 110a, 110b issue motion data A_{C} 132a and motion data A_{V} 132b, however, unlike FIG. 1A, the motion data A_{C} 132a and motion data A_{V} 132b is received directly by the portable device 114 rather than being passed indirectly to the portable device 114 via the defibrillation/monitor device 108. In this arrangement of communications, the processing circuitry of the defibrillation/monitor device 108 is freed from combining data with the motion data A_{C} 132a and motion data A_{V} 132b from the wearable devices 110a, 110b. However, the portable device 114 will now include the addition of processing capability to perform co-registration of timings of the motion data A_{C} 132a and motion data A_{V} 132b of the wearable devices 110a, 110b with the data B_{CV} 134, transferred from the defibrillator/monitor 108. Co-registering at the portable device 114, for example, may result in less refined data combination that may be achieved using the rich data set of the defibrillation/monitor device 108 prior to distillation into metrics compatible with the portable device 124.

Turning to FIG. 1C, in an example emergency scene 170, the rescuers 104, 106 are wearing the wearable devices 110a, 110b and performing chest compressions and ventilations on the patient 102. As in FIG. 1A, the wearable devices 110a, 110b issue motion data A_{C} 132a and motion data A_{V} 132b to the defibrillation/monitor device 108. However, in the emergency scene 150, the supervisor 118 and the portable computing device 114 are not included. Instead, the defibrillator/monitor 108 issues the data B_{CV} 134 directly to the remote computing system 116. Additionally, the defibrillator/monitor 108 provides the coaching or prompting data C_{C} 136a and the data C_{V} 136b to the wearable devices 110a, 110b. Although in this arrangement the defibrillation device 108 does the processing activity required to generate the data C_{C} 136a and the data C_{V} 136b, less equipment is required to provide prompting to the rescuers 104, 106. Further, without an additional communication hop, the data C_{C} 136a and the data C_{V} 136b has an even greater likelihood of reaching the rescuers 104, 106 to provide real-time feedback. However, the software of the portable computing device 114 may be easier to update with information such as preferred user interface settings and/or desired prompting styles such that the data provided to the wearable devices 110a, 110b may be customized to the preferences of individual wearers.

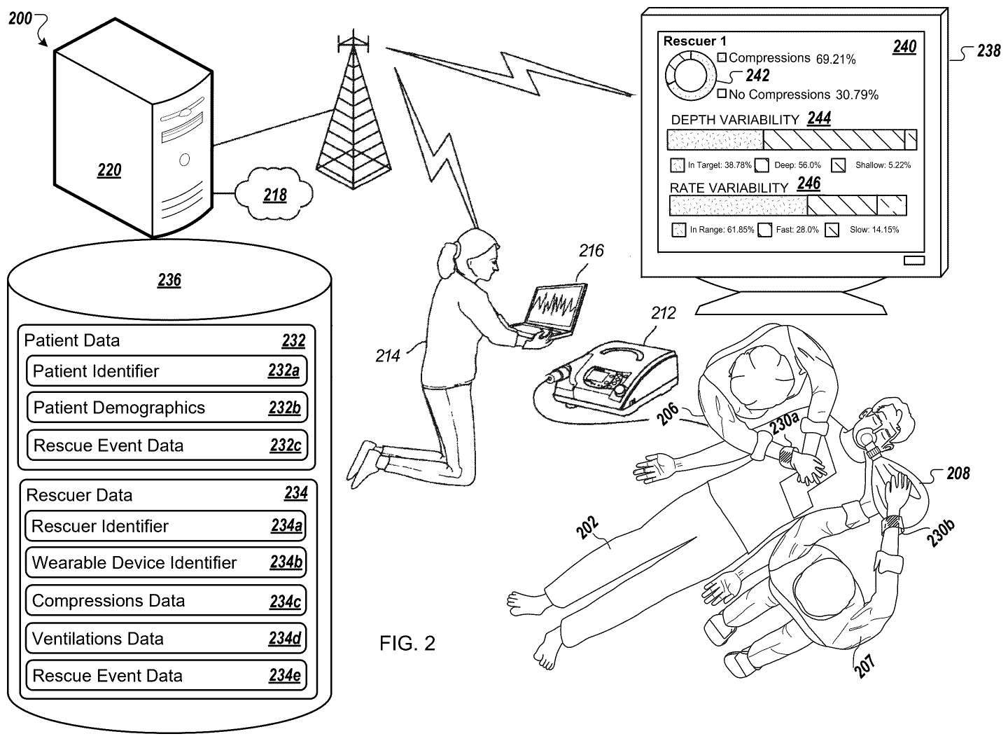

FIG. 2 illustrates an example environment 200 and medical treatment system for analyzing data collected from wearable computing devices donned by rescuers performing various roles at a medical emergency scene. In general, the environment 200 includes various portable devices for monitoring on-site care given to a victim 202. The various devices may be provided by emergency medical technicians who arrive at the scene and who provide care for the victim 202, such as emergency medical technicians 206, 207, and 214. In this example, the emergency medical technicians 206, 207, and 214 have deployed several devices and are providing care to the victim 202. The emergency medical technician 214 in this example is interacting with a computing device 216 (e.g., a touchscreen tablet, notebook computer, or laptop computer). The computing device 216 may include a graphical display by which to report information to the emergency medical technician 214. A portable defibrillator/monitor 212 is shown in a deployed state and is connected to the victim 202. In addition to providing defibrillation, the defibrillator/monitor 212 may serve as a patient monitor via a variety of sensors or sensor packages. For example, electrodes may be applied to the bare chest of the victim 202 and may be connected to the defibrillator/monitor 212, so that electrical shocking pulses may be provided to the electrodes in an effort to defibrillate the victim 202, and electrocardiogram (ECG) signals may be read from the victim 202. The defibrillator/monitor 212 may provide feedback to a rescuer via a display or via a separate computing device, such as the computing device 216 used by the emergency medical technician 214.

The defibrillator/monitor 212, in some embodiments, communicates through a wireless data connection, for example using a short-range wireless communication protocol or Wi-Fi, with the computing device 216. The defibrillator/monitor 212 may provide the computing device 216 with patient status information, such as information received through the electrode assembly, including ECG information for the victim 202. In some implementations, the defibrillator/monitor 212 sends information about the performance of chest compressions, such as depth and rate information for the chest compressions, to the computing device 216. The computing device 216, in some implementations, receives data from the other sensors associated with the victim 202, such as an airflow sensor provided with a ventilation bag 208.

In some implementations, the computing device 216 receives data from a set of wearable computing devices 230a and 230b worn by rescuers 206 and 207 respectively. The information from the wearable computing devices 230a and 230b can include the motion data A_{C} 132a and motion data A_{V} 132b described in relation to FIG. 1A.

In some embodiments, a remote computing system 220, such as a server or cloud computing platform, communicates with the computing device 216 or other devices at the rescue scene over a network 218, which may include portions of the Internet (where data may be appropriately encrypted to protect privacy). The remote computing system 220 may be part of a larger system for a healthcare organization in which medical records are kept for various patients in the system. For example, patient data 232 about the patient 202 may be associated with an identification number 232a or other identifier and stored by the remote computing system 220 in a storage region 236 (e.g., database, data warehouse, neural network, etc.) for later access. The patient data 232, in some examples, may include patient demographics 232b (e.g., age, gender, address, birth date, etc.) and rescue event data 232c (e.g., physiological data, diagnoses, interventions performed, time of event, location of event, etc.).

In some implementations, the remote computing system 220 stores rescuer data 234 that includes information associated with each rescuer in the system, such as the medical technicians 206, 207, and 214. Information about the each of the rescuers 206, 207, and 214 may then be associated with an identification number 234a and/or wearable device identifier 234b and stored by the remote computing system 220 for later access. This information can include rescue event data 234e regarding each rescue attempt in which the rescuer 206, 207, and 214 participated and their role(s) in the rescue. Additionally, the information about the rescuers 206, 207, and 214 can include information received from the wearable devices 230a, 230b worn by each rescuer 206, 207, as well as, in some embodiments, data from the defibrillator/monitor 212, ventilation device 208, other medical devices, and/or other computing devices within the environment 200. For example, the rescuer data 234 can include ventilations data 234d and/or compressions data 234c related to the ventilation and compression activities of each rescuer 206, 207, 214.

Other users may then access the data in the remote computing system 220. For example, an emergency room physician may review the data from remote computing system 220. In this manner, the remote computing system 200 may permit various portable electronic devices to communicate with each other, thereby coordinating care that is provided to a victim 202. In addition, the remote computing system 200 may allow the technician 214 and others to see raw real-time data, derived real-time data (e.g., calculated metrics), and/or historical data about a rescue attempt.

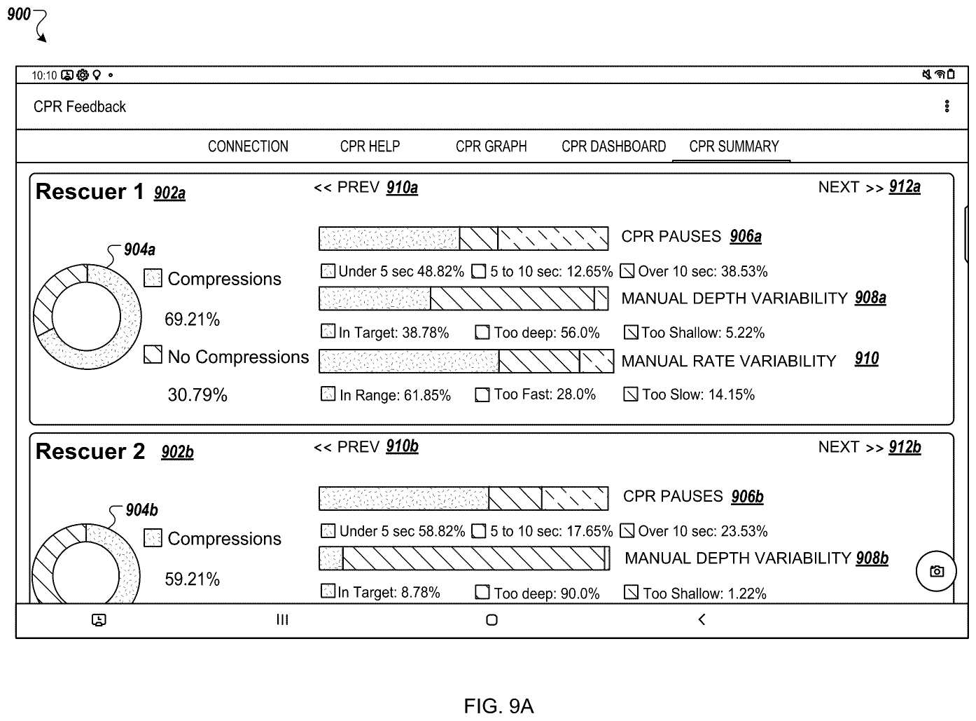

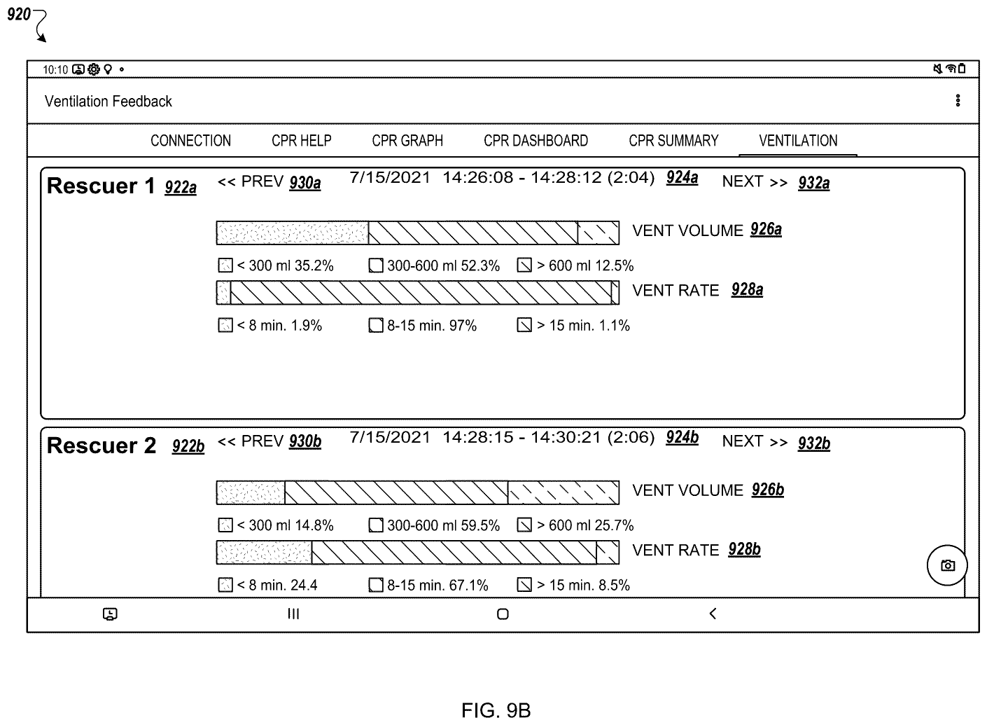

For example, as illustrated on a display 238, a synopsis of rescuer performance screen shot 240 for “Rescuer 1” is presented, including a percentage of time compressions were performed, 242, a depth variability bar graph 244, and a rate variability bar graph 246. A similar style of performance synopsis may be presented for ventilation performance, in some embodiments. Certain contents of the screen shot 240 are discussed in greater detail below in relation to FIG. 9A.

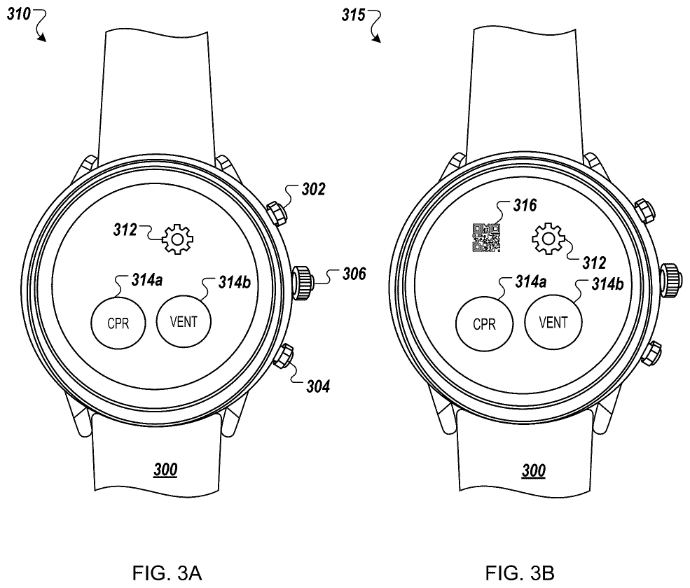

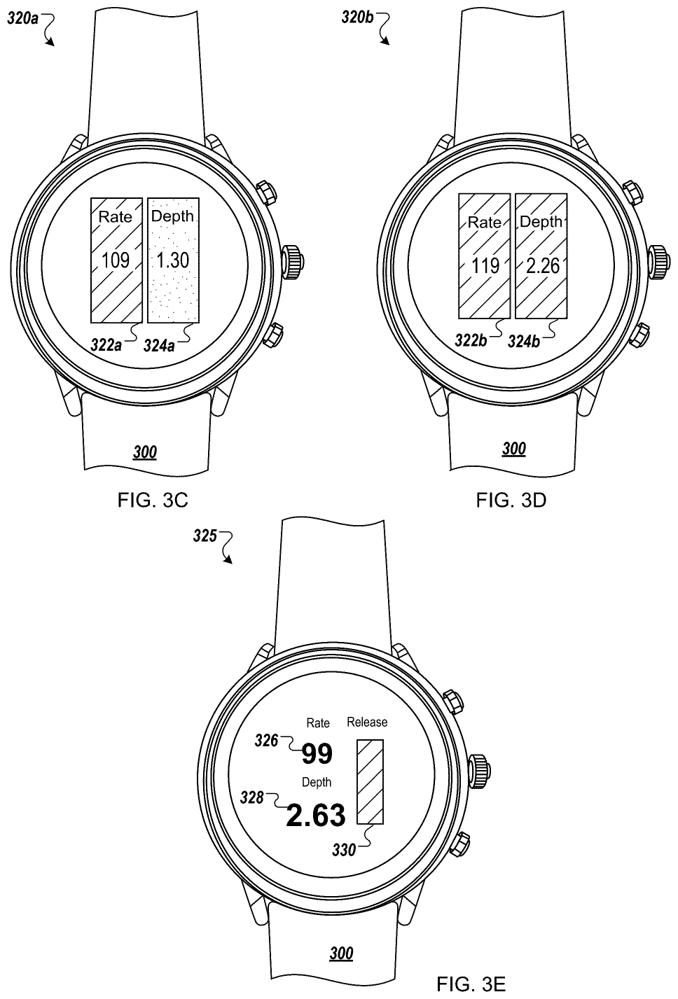

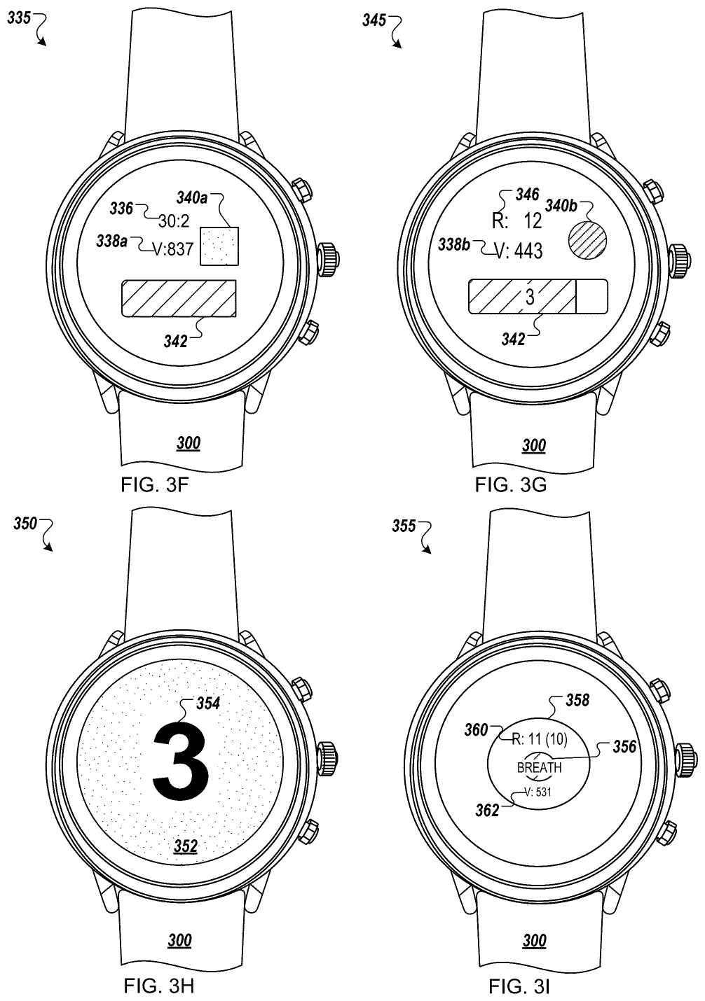

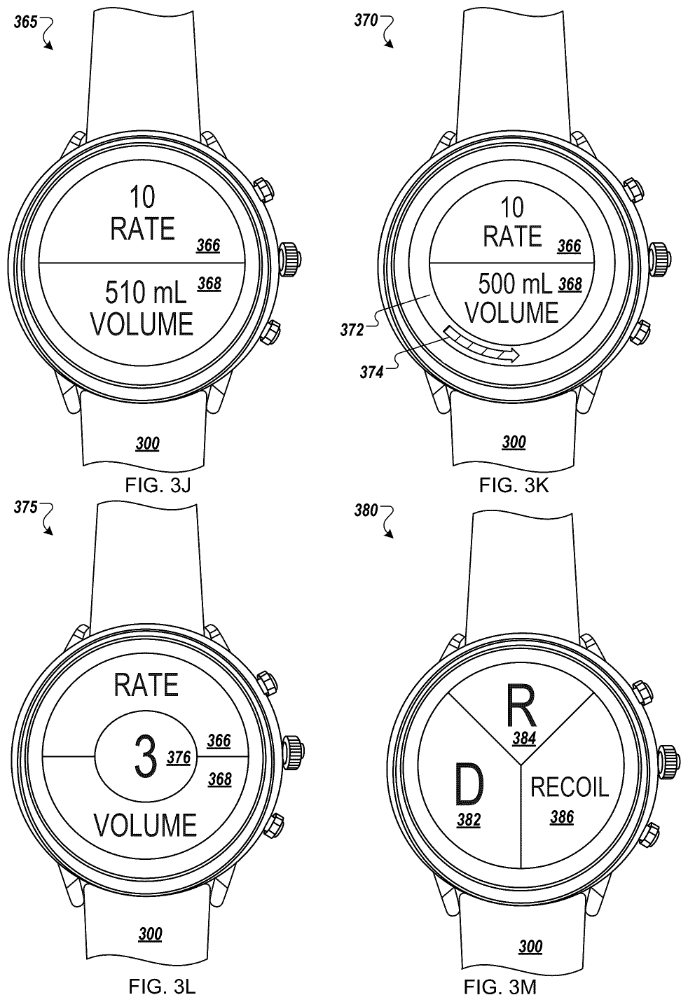

FIG. 3A through FIG. 3M illustrate example display presentations for a wearable computing device 300 configured for role-based feedback. Although each of the example display presentations are illustrated on a same wearable computing device 300, in some implementations, certain example display presentations may be provided on a smart watch, a fitness monitoring wearable device, smart glasses, a computerized augmented reality face shield or visor, a handheld portable device such as a mobile phone or tablet computer, or other portable or wearable computing device. The example display presentations, in some embodiments, are presented to the rescuers 104 and 106 of FIG. 1A through FIG. 1C on the wearable devices 110a, 110b. In some embodiments, the example display presentations are presented to the rescuers 206 and 207 on the wearable devices 230a, 230b of FIG. 2. Further, in some embodiments, portions of the example display presentations may be incorporated into user interface screens of the portable device 114 of FIG. 1A and/or the portable device 216 of FIG. 2.

Turning to FIG. 3A, the wearable computing device includes three controls, including an upper button control 302, a lower button control 304, and a dial control 306. The controls 302, 304, and/or 306 may be used to interact with the interfaces presented in FIG. 3A through FIG. 3I. For example, the upper button control 302 and lower button control 304 may be used to navigate between interfaces, while the dial control 306 may be used to select options within a given interface. In some embodiments, wearable computing devices may include more or fewer controls such as, in some examples, between one and five controls. In further embodiments, the wearable device 300 includes one or more virtual controls such as, in some examples, a touch screen, gesture recognition, and/or voice command recognition for interacting with the wearable device 300.

In some implementations, as illustrated in a first example user interface 310 of FIG. 3A, a setup icon 312 is provided to enable user control to set up parameters of the wearable device 300. In some examples, the setup mode may assist in pairing (e.g., move into a discover mode, issue a pairing signal, display a machine-readable code such as a QR code to aide in pairing, etc.), allow the user to register with the device (e.g., name, employee ID, password, biometric recognition, etc.), and/or provide options for feedback styles and/or types.

In some implementations, as illustrated in the first example user interface 310, the user is presented with mode icons 314, such as a CPR mode 314a (e.g., compressions) and a ventilation mode 314b. The mode icons 314, for example, may provide user control to initially select a feedback mode. Other potential feedback modes include a drug delivery mode, a traumatic brain injury (TBI) mode, a trauma mode, a difficulty breathing mode, a cardiologist mode, and/or a supervisor mode. Certain feedback modes may involve context-sensitive guidance and/or other information supporting decision making for the particular user of the respective mode during the emergency event. The supervisor mode, for example, may allow the user designated as supervisor to view feedback, guidance, and/or reports associated with the performance of other rescuers. Such information in the supervisor mode may be viewed in real-time during the emergency situation and/or afterward for post-case review. In a further example, an instructional or training mode may involve a specialized interface for presenting information and/or feedback to a trainee. In a TBI mode, for example, the feedback provided to the user may include graphical trend data of patient vitals such as SpO2, blood pressure (systolic and/or diastolic), and ETCO2, along with ventilation feedback as described herein.

Turning to FIG. 3B, a second example user interface 315, in some implementations, includes a machine-readable code icon 316 provided to enable a user to present a machine-readable code for pairing with another computing device or medical device, such as the defibrillation/monitor device 108 or the portable computing device 114 of FIG. 1A. The machine-readable code, in some examples, may include a data matrix barcode, quick response (QR) code, Aztec code, or color barcode used to identify the wearable computing device with another computing device. For example, upon selection of the machine-readable code icon 316, the face of the wearable computing device 302 may be substantially filled with a machine-readable code for scanning by the other computing device. The machine-readable code may be dynamic or static in nature. In some embodiments, the machine-readable code includes encrypted information relating to establishing a connection with the medical device. The computing device or medical device configured to pair with the wearable computing device 302, for example, may be configured to capture an image of the machine-readable code, decode the machine-readable code to determine the encrypted information, and decrypt the encrypted information. Decryption, for example, may be performed using a decryption key accessible by an application running on the computing device or medical device. The information, for example, may include network connectivity information such as a network identifier, a wearable computing device identifier, session information, security information, and/or other similar connection information associated with the wearable computing device and included in the encrypted information of the machine-readable code. To pair the wearable device, the computing device or medical device, for example, may establish direct or indirect connection with the wearable computing device using the connection information.

FIG. 3C through FIG. 3E illustrate example user feedback interfaces for presenting CPR feedback to a rescuer providing chest compressions. The types of user interfaces presented in FIG. 3C through FIG. 3E, in some embodiments, are user-selectable via the settings icon 312 of the example user interface 310 of FIG. 3A or the example user interface 315 of FIG. 3B. The CPR user feedback interfaces, for example, may be presented responsive to selection of the CPR mode represented by the CPR mode icon 314a of the example user interface 310 of FIG. 3A or the example user interface 315 of FIG. 3B.

FIG. 3C and FIG. 3D present a first example CPR feedback user interface 320a, 320b including a compression rate indicator 322a, 322b and a compression depth indicator 324a, 324b. Beginning with the compression rate indicator, a bar 322a, 322b is marked with a current compression rate (e.g., 109 and 119, respectively). Further, the font (e.g., “rate” and/or “109”) and/or the rendering of the bar 322a, 322b may be indicative of sufficiency of the present compression rate. As illustrated the font style is the same and the fill style (e.g., representative of color) is the same between FIG. 3C and FIG. 3D.

However, turning to the compression depth indicator 324a, 324b, a fill style (e.g., color) of the compression depth indicator bar 324a of FIG. 3A differs from the fill style of the compression depth indicator bar 324b of FIG. 3D. As illustrated the compression depth indicator 324a represents a “Depth” of “1.30” (e.g., insufficient), while the compression depth indicator 324b represents a “Depth” of “2.26” (e.g., sufficient).

Compression rates and/or compression depths, in some embodiments, are visually represented in at least two separate colors based on ranges of rates and/or depths. For example, compression rate may be designated as “slow”, “sufficient,” or “fast,” (e.g., orange, green, yellow) and color markings or other visual indication may be adjusted appropriately. In another example, compression rate may be designated as “outside sufficient range” or “inside sufficient range” (e.g., green or red). In a further example, compression rate may be designated as “far outside range”, “dropping outside range”, or “sufficient”, with either three (e.g., red, yellow, green) options for indication or five options for indication (e.g., to differentiate visibly between too slow or too fast). Further to this example, the bar fill may designate which range (e.g., partially filled representing slower than sufficient, filled representing faster than sufficient). In an illustrative example, a green depth indicator may indicate that compression depth for an ongoing or most recent compression is within the target range, while a yellow depth indicator may indicate that compression depth is just outside of target range, and an orange or red depth indicator may indicate that compression depth is further outside of the target range as compared to the compression depth indicated by the yellow depth indicator.

Similarly, turning to compression depth, overcompression/undercompression may be dealt with in a similar manner or in two different manners (e.g., sufficient compression or not sufficient compression; undercompression, sufficient compression, or overcompression, etc.). Visual indications may be applied in a similar fashion as described in relation to compression rate.

Turning to FIG. 3E, a second example CPR feedback user interface 325 includes a numeric indication of rate 326 (e.g., 99), a numeric indication of depth 328 (e.g., 2.63), and a release timing indicator 330. The numeric indications of rate 326 and depth 328, for example, may be differentiated by font style and/or color to indicate values outside a range of sufficiency. The release timing indicator 330, in some embodiments, changes color at a depth corresponding to sufficient, full compression. The release timing indicator 330, for example, may be yellow during the downward push to sufficient depth, then turn green to signal release. If the chest has been overcompressed or if the release velocity from the chest is insufficient, the release timing indicator 330 may turn red. Instead of or in addition to the release timing indicator 330, in some embodiments, an audible and/or haptic signal may be provided to prompt the release of compression.

Turning to FIG. 3M, a third example CPR feedback user interface 380 of the wearable device 300 includes a “pie graph” style design in which three wedges 382, 384, and 386 are laid out to represent metrics related to compression delivery. The user interface 380 includes a depth wedge 382 marked “D” representing sufficiency of depth of compression, a rate wedge 384 marked “R” representing sufficiency of rate of compression, and a recoil wedge 386 marked “RECOIL” representing velocity and/or completeness of release from the patient's thorax after compression. Each of the wedges 382, 384, and 386 may be individually filled with a color and/or pattern indicative of relative sufficiency of the representative metric. In an illustrative example, the rate wedge 384 may be colored green to indicate that compression rate is within target range, while the recoil wedge 386 may be colored yellow to indicate that compression release was a little too slow, and the depth wedge 382 may be colored red to indicate that the compression depth was substantially outside of the target range.

FIG. 3F through FIG. 3L illustrate various user interfaces for presenting ventilation related feedback to a rescuer using the display of a wearable computing device. The types of user interfaces presented in FIG. 3F through FIG. 3L, in some embodiments, are user-selectable via the settings icon 312 of the example user interface 310 of FIG. 3A or the example user interface 315 of FIG. 3B. The ventilation user feedback interfaces, for example, may be presented responsive to selection of the ventilation mode represented by the ventilation mode icon 314b of the example user interface 310 of FIG. 3A or the example user interface 315 of FIG. 3B.

Turning to FIG. 3F and FIG. 3G, in some implementations, an example user interface 335 and an example user interface 345, respectively, include a time to ventilation delivery bar 342 for visually monitoring a countdown to the next ventilation delivery, and a ventilation volume sufficiency icon 340a, 340b providing easy-to-read visual feedback related to the volume delivered with the most recent ventilation. The ventilation delivery bar 342, as illustrated in FIG. 3B, includes a numeric indicator (e.g., “3”), indicating a number of seconds remaining prior to ventilation delivery. The ventilation volume sufficiency icon 340a, 340b, in some embodiments, presents a color and/or shape associated with sufficiency of the volume of air delivered. For example, the ventilation volume sufficiency icon 340a, 340b may be green for sufficient, yellow for under-delivery, and red for over-delivery. The ventilation volume sufficiency icon 340a, 340b may adjust in color during delivery of air to the victim.

As illustrated, the example user interfaces 335, 345 of FIG. 3F and FIG. 3G include a tidal volume (e.g., in milliliters) metric 338 indicating a volume of the most recently delivered ventilation. As illustrated, the tidal volume metric 338a of FIG. 3F is 837 mL, and the tidal volume metric 338b of FIG. 3G is 443 mL.

Turning to FIG. 3F, in some implementations, the user interface 335 of the wearable computing device 300 includes a mode indicator 336. The mode, for example, may identify a ratio of compressions to ventilations. The ratio may differ, for example, based on an age of the patient. As illustrated, the mode indication 336 reads 30:2 (e.g., thirty compressions followed by two ventilations).

Turning to FIG. 3G, in come implementations, the user interface 345 of the wearable computing device 300 includes a rate of ventilations indicator 346 representing a rate of ventilations per minute (e.g., “12”).

As illustrated in FIG. 3H, in some implementations, an example user interface 350 of the wearable device 300 includes a countdown value 354 (e.g., “3”) and a background color or fill 352 indicating sufficiency of fill.

In another example user interface 355 of FIG. 3I, in some implementations, the wearable device 300 presents ventilation feedback using a multi-ring format with an inner ring 356 and an outer ring 358. The inner ring 356 may expand outwardly to the outer ring 358 to represent sufficiency of delivery of a volume of air to the patient during each ventilation. Further, the inner ring 356 may be filled with a color and/or pattern indicative of sufficiency of ventilation delivery.

The user interface 355, in some implementations, includes a rate of ventilations indicator 360 representing a rate of ventilations per minute (e.g., 11 out of a target 10), and a tidal volume metric 362 (e.g., 531 mL).

Turning to FIG. 3J through FIG. 3L, in some implementations, the wearable device 300 presents ventilation feedback in a user interface 365 having split screen with a rate (e.g., 10) represented in an upper half 366 of the display region and a volume (e.g., 510 mL) represented in a lower half 368 of the display region. Further, each of the upper half 366 of the display region and the lower half 368 of the display region may be filled with a color and/or pattern indicative of sufficiency of ventilation delivery. For example, both the upper 366 and lower 368 display regions may be filled with a same color/pattern (e.g., green, indicating sufficiency of both rate and volume) or a color/pattern of the upper half 366 may be different than a color/pattern of the lower half 366 (e.g., upper yellow, indicating a rate just outside of the sufficiency range for ventilation rate and lower red, indicating a volume further outside of the sufficiency range for ventilation volume).

In FIG. 3K, the wearable device 300, in some implementations, a user interface 370 presents the split screen feedback layout of FIG. 3J, but the upper half 366 and the lower half 368 of the display region have been reduced in size to present an outer ring 372 encircling the upper half 366 and lower half 368 of the display region. The outer ring, for example, may represent a time to next ventilation, where an arrow 374 within the outer ring 372 expands to fill more of the outer ring 372 as time gets closer to the next ventilation time. The arrow 374, for example, may be presented in a contrasting color to a fill of the outer ring 372. Rather than an arrow, in other embodiments, a gradient or single tone fill may gradually around the outer ring 372 (e.g., clockwise or counterclockwise) to present a visual countdown until the next ventilation.

Turning to FIG. 3L, in some implementations, a user interface 375 of the wearable device 300 presents the split screen feedback layout of FIG. 3J with an inner circle 376 presenting a visual countdown until the next ventilation. As illustrated, the inner circle 376 is marked with the number 3, representing three seconds until the next ventilation. The inner circle 376, in some embodiments, may change in fill and/or color as the time to ventilation delivery approaches.

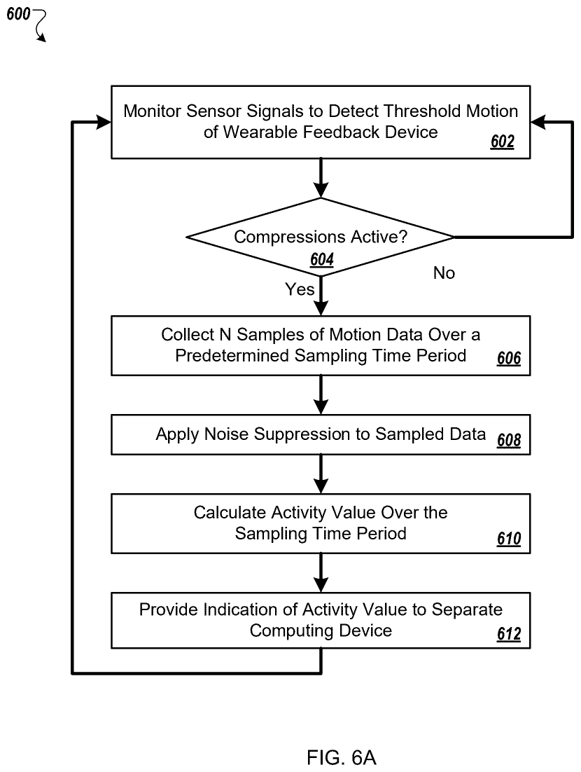

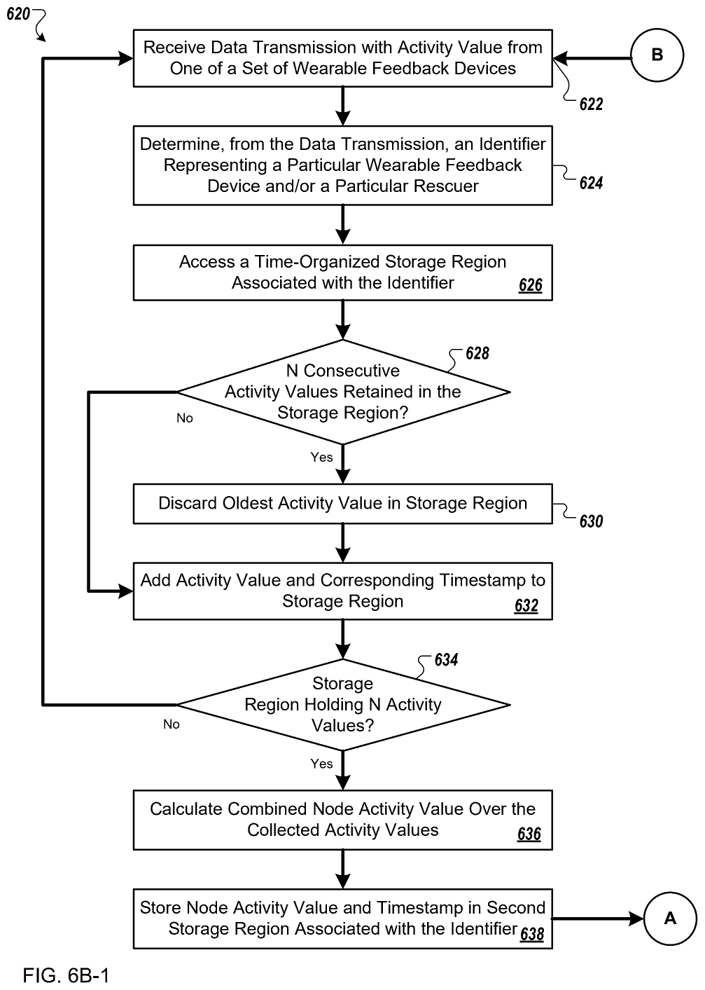

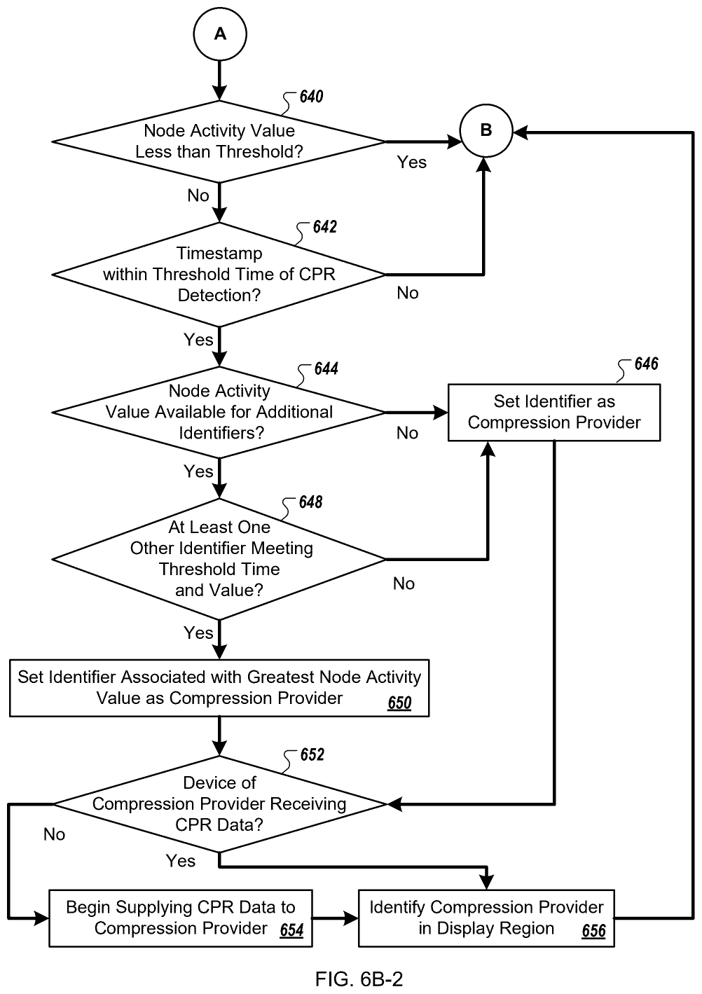

FIG. 6A is a flow chart of an example method 600 for calculating an activity value corresponding to motions of a wearable rescuer feedback device that are indicative of the type of activity the wearer is performing. The method 600, for example, may be performed by the wearable computing devices 110a and 110b of FIG. 1A through FIG. 1C and/or by the wearable computing devices 282a and 282b of FIG. 2. In some embodiments, portions of the method 600 may be performed by a device receiving data from the wearable computing devices, such as the defibrillation/monitor device 108 of FIG. 1A and FIG. 1C, the portable computing device 114 of FIG. 1B, the portable computing device 216 of FIG. 2, or the portable defibrillator/monitor 212 of FIG. 2.

In some implementations, the method 600 begins with monitoring sensor signals to detect threshold motion of a wearable rescuer feedback device (602). In some implementations, the threshold motion is calibrated based on typical behaviors of a wearer and/or typical behaviors of a wearer during a particular type of activity. The threshold motion, for example, may include a threshold acceleration, motion over time in at least two directions, and/or a motion outside of a motion profile of the wearer considered to be “at rest” and/or “walking.” The wearable computing device (e.g., device 110a, 110b, 282a, and/or 282b), for example, may monitor sensor data collected by one or more sensors of the wearable computing device. The sensors, in some examples, can include an accelerometer, a gyroscopic sensor, and/or a magnetic sensor. Further to the examples, detecting motion can include detecting acceleration in one or more directions via sensor signals sensed by an accelerometer, detecting a gyroscopic progression of sensor signals sensed by a gyroscopic sensor (e.g., angular velocities and/or orientations) and/or detecting one or more magnetic field measurements from sensor signals sensed by a magnetic sensor.

If chest compressions are determined to be active (604), in some implementations, a series of N samples of motion data is collected over a predetermined sampling time period (606). To determine chest compressions are active, for example, an indication may be obtained from a medical device, a compression depth monitoring device, or a separate computing device. For example, as illustrated in FIG. 1A, the indication of active chest compressions may be provided to the wearable devices 110a, 110b in the data 136a, 136b from the portable computing device 114. In another example, in relation to FIG. 1C, the indication of active chest compressions may be provided to the wearable devices 110a, 110b in the data 136a, 136b from the defibrillation/monitor device 108. In some embodiments, the wearable computing device (e.g., device 110a, 110b, 282a, and/or 282b), collects the samples of motion data. In other embodiments, the wearable computing device provides the motion data to a separate device, such as the defibrillation/monitor device 108, the portable computing device 114, the portable computing device 216, or the portable defibrillator/monitor 212, for collection.

In some embodiments, each motion data sample is gathered over a sample time period of less than half a second, between half a second and a second, or up to about one second. The motion data sample may be an acceleration data sample, for example based on signals of an accelerator element of the wearable device. The motion data samples may be collected over a period of time of between three and five seconds, at least five seconds, or up to ten seconds. In another example, motion data samples may be continuously collected while compressions are active and/or while sensor signals detect threshold activity of the wearable feedback device. In some implementations, motion data samples are collected over the period of time on a periodic basis. For example, there may be less than a half of a second between samples (e.g., about 200 msec, about 300 msec, etc.), at least a half of a second between samples, a second between samples, or between a second and two seconds between consecutive samples. The periodicity of samples, in some examples, may be based on battery levels of the wearable device.

A number of total motion data samples, in some embodiments, is at least three, between three and five, or up to ten data samples. The motion data samples may be collected as a time progression over time, where a total number of motion data samples stored at one time is capped to a threshold number N. For example, a motion data FIFO may store up to N (e.g., three, five, ten, etc.) most recent motion data samples collected. The threshold number of motion data samples may relate to a threshold period of time over which samples are collected. In some examples, over two seconds of data is collected, at least five seconds of data is collected, or under ten seconds of data is collected.

In some implementations, noise suppression is applied to the sample data (608). The noise suppression, for example, may remove data outside of a typical range or that otherwise appears to be errant signals not indicative of the activities of the wearer of the wearable device. In some embodiments, the wearable computing device (e.g., device 110a, 110b, 282a, and/or 282b), applies noise suppression. In other embodiments, a separate device, such as the defibrillation/monitor device 108, the portable computing device 114, the portable computing device 216, or the portable defibrillator/monitor 212, applies noise suppression to the sample data.

In some implementations, an activity value is calculated over the sampling period (610). The activity value is calculated to be representative of a user's activities (e.g., movements, exertions, forces, etc.) over the sample time period. The activity value, for example, may be calculated to match motion data to rescue activities, such as compression delivery or ventilation delivery. The activity value may be calculated in part by identifying a dominant frequency of compression rate. In one example, calculating the activity value may include applying pattern matching to the time progression of values. In a particular example, the activity value may be calculated as the root-mean-square (RMS) value of acceleration data. The activity value, for example, may be calculated across multiple directions of motion (e.g., axes of acceleration). In some embodiments, the wearable computing device (e.g., device 110a, 110b, 282a, and/or 282b), calculates the activity value. In other embodiments, a separate device, such as the defibrillation/monitor device 108, the portable computing device 114, the portable computing device 116, or the portable defibrillator/monitor 212, calculates the activity value.

In some implementations, an indication of the activity value is provided to a separate computing device (612). The separate computing device, in some examples, may be the portable computing device 114 of FIG. 1A, the defibrillation/monitor device 108 of FIG. 1A through FIG. 1C, the portable defibrillator/monitor 212 of FIG. 2, and/or the computing device 216 of FIG. 2. For example, the wearable computing devices may provide the indication of the activity value to a defibrillator/monitor or to a portable computing device. In another example, a defibrillator/monitor may provide the indication of the activity value to a portable computing device. The indication of the activity value, for example, may include the activity value, a rounded version of the activity value, or a characterization of the activity value (e.g., below a first threshold, above a second threshold, etc.). For example, the activity value may be compared to a threshold corresponding to chest compression activity. In some embodiments, the indication of the activity value is provided along with additional information such as, in some examples, an identifier of the wearable device, an identifier of the rescuer, a timestamp, a battery level, and/or a setting of the wearable device.

The method 600, in some implementations, repeats during periods of active chest compressions (604).

Although described as a particular series of operations, in some embodiments, more or fewer steps may be included in the method 600. For example, motion data may be collected (606) without indication of compressions being active (604). In another example, the activity value may be calculated over the most recent N samples collected and in response to a request from the separate computing device. In further embodiments, certain steps of the method 600 may be performed in a different order or in parallel. For example, noise suppression may be applied to each sample prior to storage. Other modifications of the method 600 are possible while remaining within the scope and extent of the method 600.

FIG. 6B-1 and FIG. 6B-2 illustrate a flow chart of an example method 620 for applying activity values corresponding to motions of a set of wearable computing devices to determine which wearable computing device is donned by a rescuer performing chest compressions. The method 620, for example, may be performed by the portable computing device 119 of FIG. 1A, the defibrillation/monitor device 108 of FIG. 1A through FIG. 1C, and/or the computing device 216 of FIG. 2.

In some implementations, the method 620 begins with receiving a data transmission with an activity value from one of a set of wearable feedback devices (622). The wearable feedback devices, for example, may be the wearable devices 110 of FIG. 1A through FIG. 1C or the wearable devices 230 of FIG. 2. The activity value, for example, may have been calculated using the method 600 of FIG. 6A.

In some implementations, an identifier representing a particular wearable feedback device and/or a particular rescuer is determined from the data transmission (624). The identifier, for example, may be used to differentiate activity values supplied by each of the wearable feedback devices. In another example, a traffic identifier (e.g., identification of a wireless communication connection) may be used to differentiate activity values.

In some implementations, a time-organized storage region associated with the identifier is accessed (626). The time-organized storage region, for example, may be a FIFO storage region. In some embodiments, each entry of the time-organized storage region is associated with a timestamp. The timestamp, in some examples, may be a timestamp included in the transmission of the activity value, a time of receipt of the activity value, or a time of storage of the activity value. For example, in the circumstance that the activity values are provided by the defibrillation/monitor device 108, the time of receipt by the defibrillation device 128 may be used rather than a time of transfer by the wearable device.

In some implementations, if there is a threshold number N of consecutive activity values retained in the storage region (628) associated with the identifier, the oldest activity value in the storage region is discarded (630). In this manner, the N most recent activity values may be retained by the method 620.

In some implementations, the activity value and corresponding timestamp are added to the storage region (632). For example, the activity value and associated timestamp (or a current time stamp) may be stored to the storage region.

If the storage region does not yet hold the threshold number N of activity values (636), in some implementations, the method 620 returns to waiting to receive another data transmission with an activity value (622).

Otherwise, in some implementations, if the storage region now holds the threshold number N of activity values (634), a combined node activity value is calculated over the collected activity values (636). The combined node activity value, for example, may include summing the node activity values, applying weights to the activity values, and/or discarding one or more activity values. For example, the activity values may be weighted such that those activity values having timestamps closest in time to delivery of a chest compression are given a stronger weight than those activity values corresponding to a pause between consecutive chest compressions. In a particular example, the combined node activity value may be calculated as an average or weighted average of the N activity values.

In some implementations, the node activity value and a corresponding timestamp are stored in a second storage region associated with the identifier (638). The timestamp, in some examples, may be a current timestamp, the timestamp associated with the largest activity value of the N activity values, or the timestamp associated with the last received (e.g., most recent) activity value of the N activity values.

Turning to FIG. 6B-2, if the node activity value is less than a threshold (640), in some implementations, the method 620 returns to waiting to receive another data transmission with an activity value (622). For example, the node activity value may be determined to be less than a threshold activity associated with active chest compressions.

In some implementations, if the timestamp of the node activity value is not within a threshold time of CPR detection (642), the method 620 returns to waiting to receive another data transmission with an activity value (622). For example, a time difference between a time of receipt of indication of compressions and a timestamp of the node activity value may be calculated to determine that they are within a predetermined period of time. In some examples, the predetermined period of time may be under 1 second, under 3 seconds, under 5 seconds, between 5 and 10 seconds, between 10 and 15 seconds, or up to 30 seconds.

In some implementations, if no node activity value(s) is available for additional identifiers associated with different wearable devices and/or rescuers (644), the identifier is set as the compression provider role (646). For example, if other wearable devices were not reporting activity within the timeframe, then the wearable device is assumed to be worn by the rescuer performing chest compressions. In some cases, when other wearable devices are not reporting activity within the timeframe, then the wearable device having some activity reaching a comparably lower threshold than usual when multiple wearable devices reporting activity may be assumed to be worn by the rescuer performing chest compressions.

If, instead, there are other node activity value(s) stored, in some implementations, it is determined whether at least one other identifier is within the threshold node activity value and within the threshold time of CPR detection (648). These determinations may be made, for example, as described in relation to operations 640 and 642, above.

If no other identifier meets the threshold time and/or the threshold value (648), in some implementations, the identifier is set as the compression provider (646), and the method 620 returns to waiting to receive another data transmission with an activity value (622). Conversely, if one or more identifiers meet the threshold time and the threshold value (648), in some implementations, the identifier associated with the greatest node activity value is set as compression provider (650). In some examples, the node activity values may be compared directly, or the node activity values may each be weighted in respect to temporal proximity to chest compression delivery prior to comparison.

Once the compression provider has been identified (646 or 650), in some implementations, it is determined whether the device of the compression provider is currently receiving CPR data (652). For example, compression feedback may already be presented to the user of the wearable device based on the device being initially set to the compression role.

If the device is not receiving compression data (652), in some implementations, compression data begins to be supplied to the device of the compression provider (654). For example, the defibrillation/monitor device 108 or portable computing device 114 of FIG. 1A, or computing device 216 of FIG. 2, may supply compression data to the wearable device. In another example, although the compression data may be supplied to the wearable device, the wearable device may not be set to present compression-related feedback to rescuer. In this circumstance, an operation mode of the wearable device may be set to presenting compression-related feedback.

In some implementations, the compression provider is identified in a display region (656). In some examples, a supervisor may review information marked with the identity of the rescuer performing chest compressions on a portable computing device such as the portable device 114 of FIG. 1A or the computing device 216 of FIG. 2, or a medical device, such as the defibrillation/monitor device 108 of FIG. 1A, may present the identity of the rescuer performing compressions on a user interface screen.

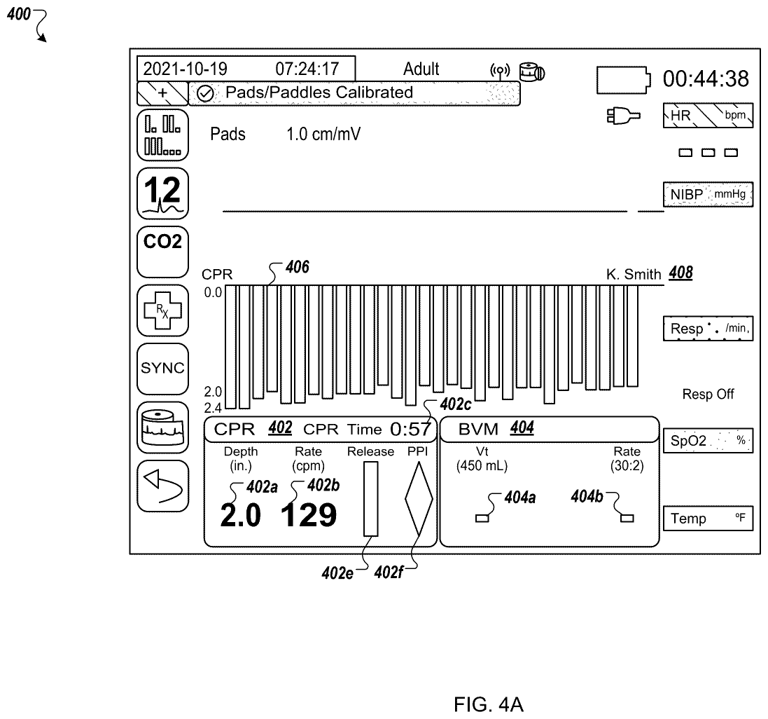

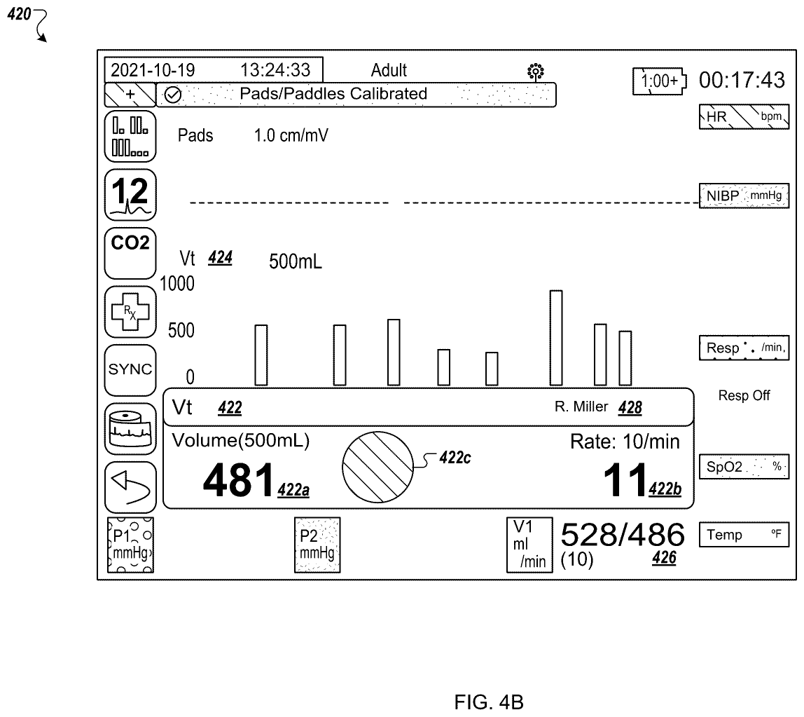

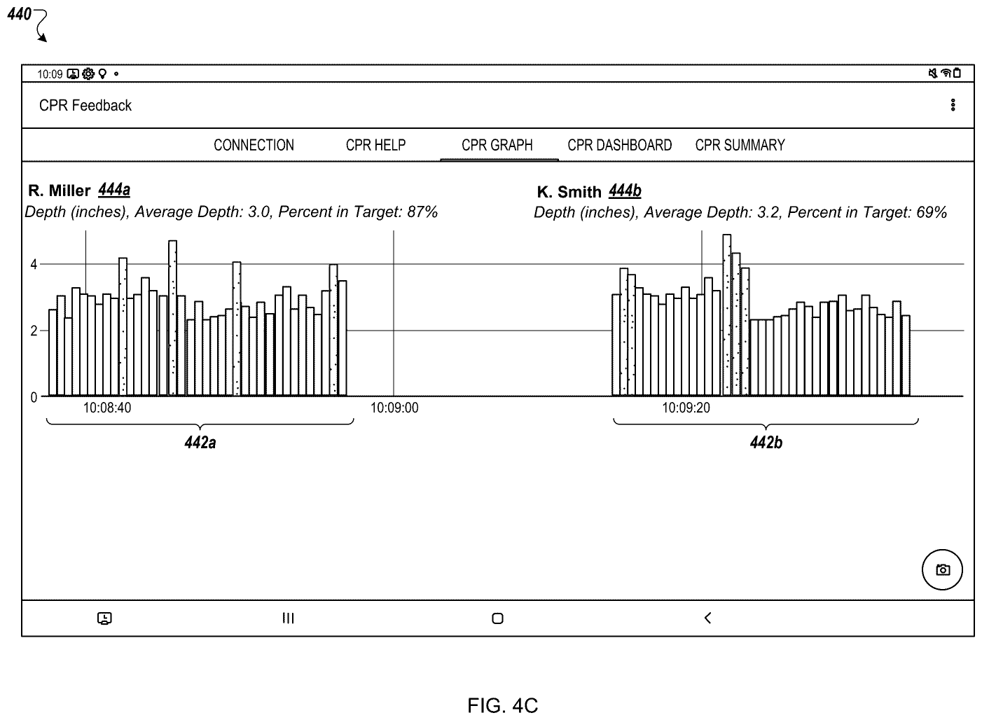

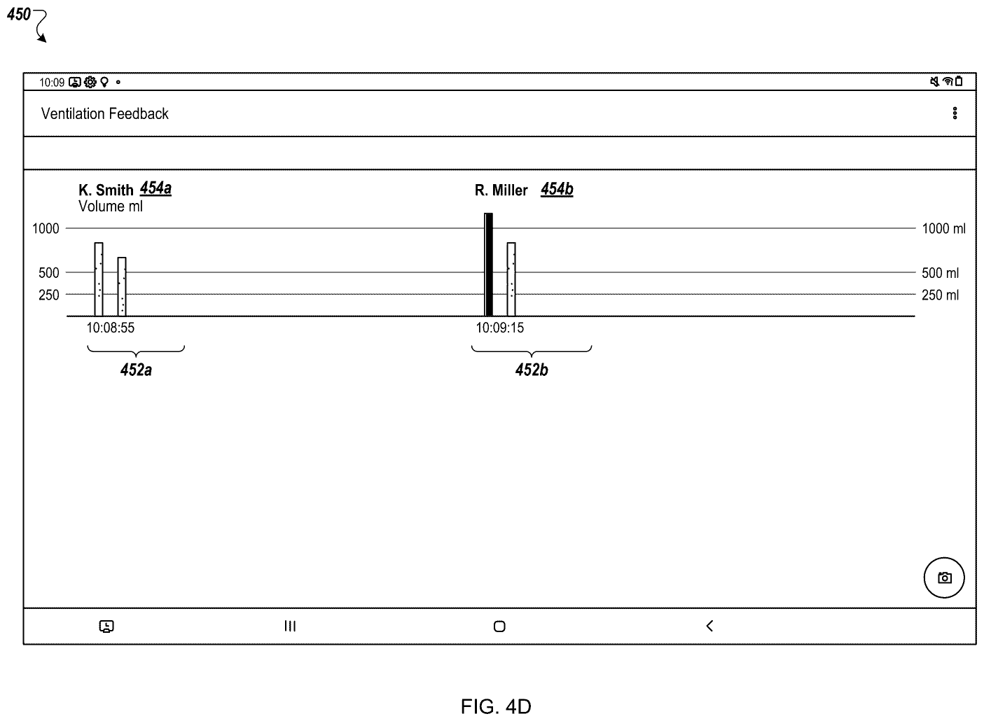

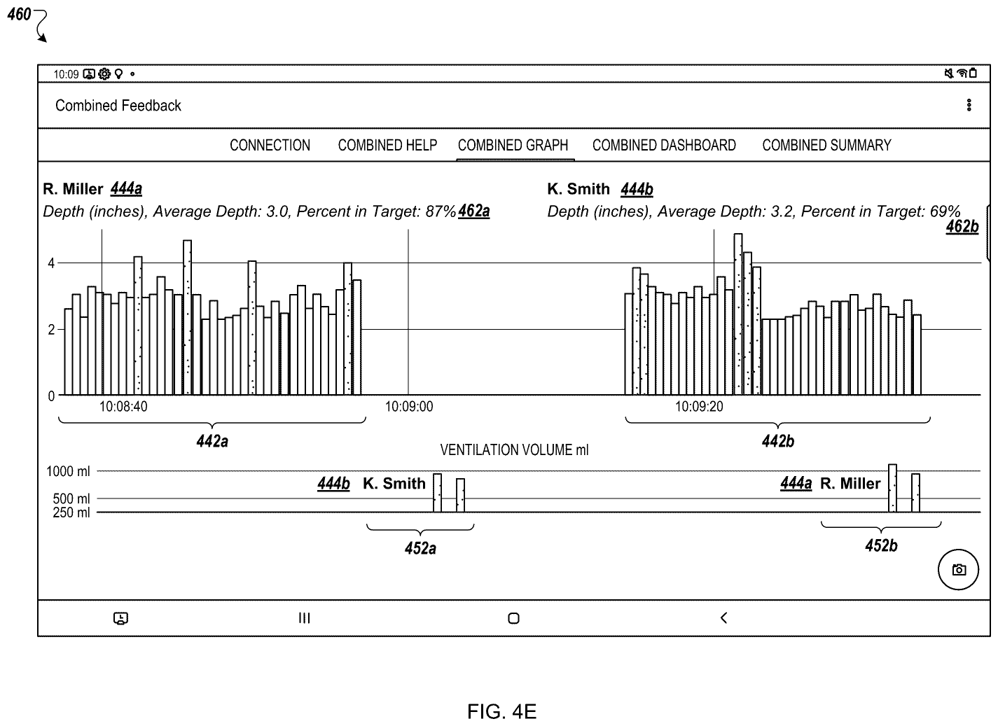

FIG. 4A through FIG. 4E illustrate example user interface screens for reviewing feedback related to a past or ongoing rescue effort. Each user interface screen includes data corresponding to at least one type of rescue role (e.g., chest compression metrics and/or ventilation metrics). The user interface screens, in some examples, may be presented by the defibrillation/monitor device 108 of FIG. 1A through FIG. 1C, the portable computing device 114 of FIG. 1A and FIG. 1B, the networked computing system 116 of FIG. 1C, the portable defibrillator/monitor 212 of FIG. 2, the portable computing device 216 of FIG. 2, and/or the remote computing system 238 of FIG. 2. The style and/or depth of content of each of the user interface screens may vary depending upon the end user device.

Turning to FIG. 4A, in some implementations, an example screenshot of a user interface screen 400 presents CPR-related feedback including CPR metrics 402 and ventilation metrics 404. The user interface screen 400, for example, may be presented on a medical device or portable screen at the scene of a medical emergency, such as the defibrillation/monitor device 108 of FIG. 1A through FIG. 1C or the portable defibrillator/monitor 212 of FIG. 2. In another example, the user interface screen 400 may be a replica or contain similar but not identical layout and information as the screen of a medical device, for example presented on a portable computing device such as the portable computing device 114 of FIG. 1A and FIG. 1B or the portable computing device 216 of FIG. 2. In this example, the replica screen may be provided for ease of review of the metrics presented by the medical device without needing to crowd around a small screen.

The CPR metrics 402 of the user interface screen 400, in some examples include a depth indication 402a (e.g., 2.0 inches), a rate indication 402b (e.g., 129 compressions per minute), a length of time since start of compressions 402c, a release indicator 402d coaching timing and/or sufficiency of compression release, and a Perfusion Performance Indicator (PPI) 402e graphically representing sufficiency of both depth and rate of compressions. The ventilation metrics 404 include a volume 404a and a rate (compressions to ventilations) 402b.

In some implementations, the user interface screen 400 presents a compression delivery graph 406 of depth of compressions over time. Further, a current rescuer 408 performing compressions (e.g., “K. Smith”) may be indicated in relation to the compression delivery graph 406.

Turning to FIG. 4B, in some implementations, an example screenshot of a user interface screen 420 presents ventilation-related feedback including ventilation metrics 422 and a ventilation delivery graph 424 graphing tidal volume over time (e.g., from 0 to 500 mL). Similar to FIG. 4A, the user interface screen 420 may be presented by a medical device and/or may be provided at a portable computing device as a proxy to the user interface screen of the medical device. The ventilation metrics 422, in some examples, may include a tidal volume 422a (e.g., “481” out of a target 500 mL), a ventilation rate 422b (e.g., “11” out of a target of 10/minute), or a ventilation sufficiency icon 422c. Further ventilation metrics may include a high tidal volume (e.g., “528”) and low tidal volume (e.g., “486”) of the most recent (e.g., “10”) ventilations 426.

In some implementations, the ventilation delivery graph 424 includes a bar graph representing the past X time interval and/or the past Y ventilation deliveries. The graph 424 may represent only those ventilations provided by a particular rescuer 428 (e.g., “R. Miller”).

Turning to FIG. 4C, in some implementations, a screen shot 440 presents compression metrics collected over time. The screen shot 440, for example, may be presented on the portable computing device 114 of FIG. 1A and FIG. 1B, the portable computing device 216 of FIG. 2, and/or the remote computing device 238 of FIG. 2. As illustrated, the screen shot 440 includes a graph of compression depth (e.g., in inches) over time, including a first graph segment 442a corresponding to a first rescuer 444a delivering chest compressions and a second graph segment 442b corresponding to a second rescuer 444b delivering chest compressions for a time period after the first rescuer 444a. Certain bars of the graph 442 are marked with a different fill, designating a visual presentation related to over-compression of the patient for each marked compression. The over-compressions, for example, may be highlighted in a different color or fill.

As shown in FIG. 4D, in some implementations, a screen shot 450 presents ventilation metrics (e.g., ventilation volume and timing) collected over time. The screen shot 450, for example, may be presented on the portable computing device 114 of FIG. 1A and FIG. 1B, the portable computing device 216 of FIG. 2, and/or the remote computing device 238 of FIG. 2. As illustrated, the screen shot 450 includes a first graph segment 452a corresponding to a first rescuer 454a delivering ventilations and a second graph segment 452b corresponding to a second rescuer 454b delivering ventilations for a time period after the first rescuer 454a. Certain bars of the graph 452, some embodiments, are marked with a different fill, designating a visual presentation related to sufficiency of ventilation, such as whether the volume delivered to the patient for each marked ventilation is within a target range, close to the target range, or outside of the target range.

Turning to FIG. 4E, in some implementations, a screen shot 460 presents both compression metrics and ventilation metrics collected over time. The screen shot 460, for example, includes the compression metrics of the screen shot 440 of FIG. 4C as well as the ventilation metrics of the screen shot 450 of FIG. 4D. The screen shot 460, for example, may be presented on the portable computing device 114 of FIG. 1A and FIG. 1B, the portable computing device 216 of FIG. 2, and/or the remote computing device 238 of FIG. 2. As illustrated, the first compression graph segment 442a corresponds to the first rescuer 444a delivering chest compressions and the second compression graph segment 442b corresponds to the second rescuer 444b delivering chest compressions for a time period after the first rescuer 444a. In addition to the graph 442, for each rescuer 444a, 444b, synopsis metrics 462a, 462b provide an average depth (e.g., 3.0, 3.2 consecutively) and a percentage of compressions within the target depth range (e.g., 87%, 69% consecutively).

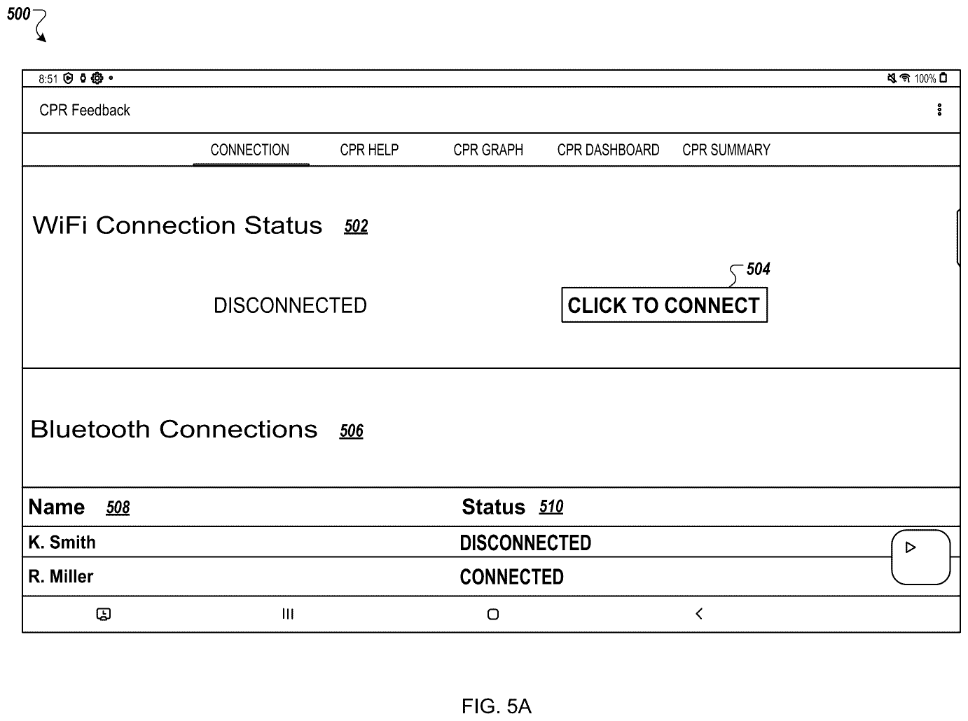

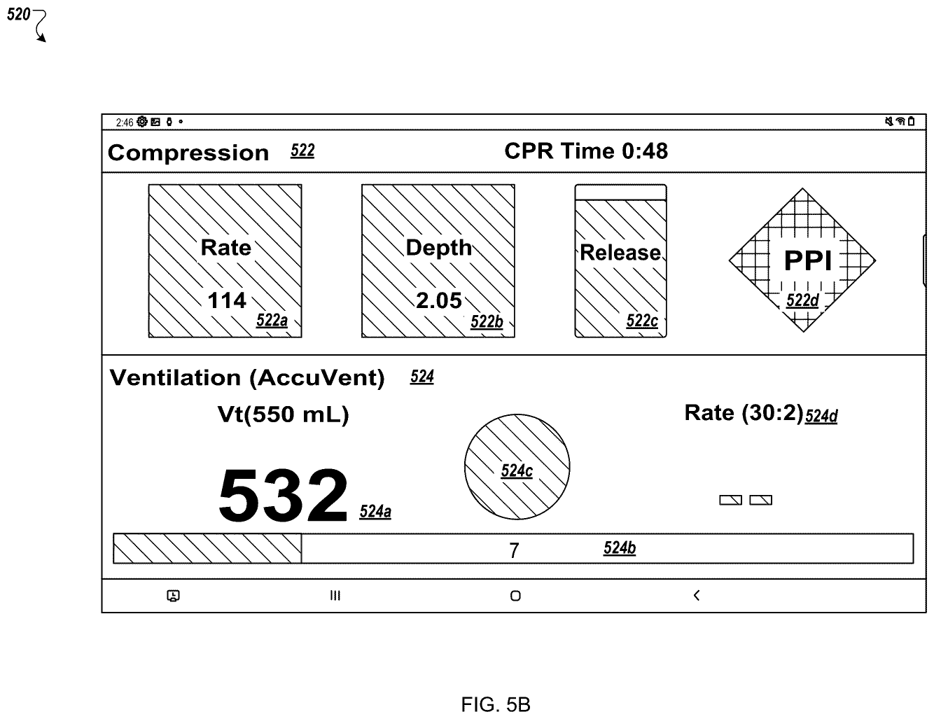

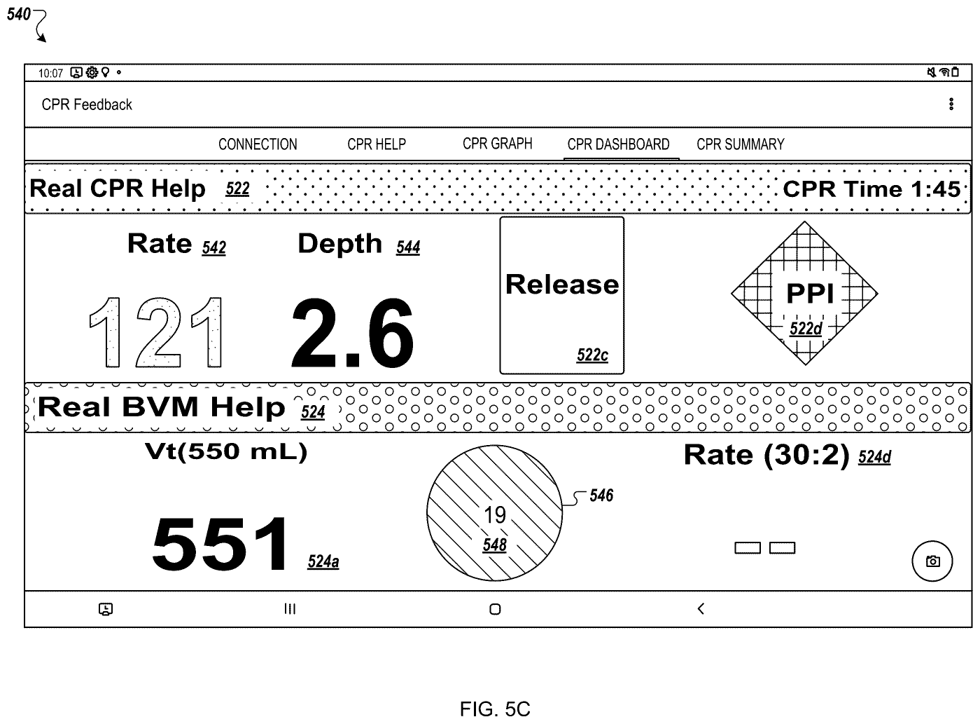

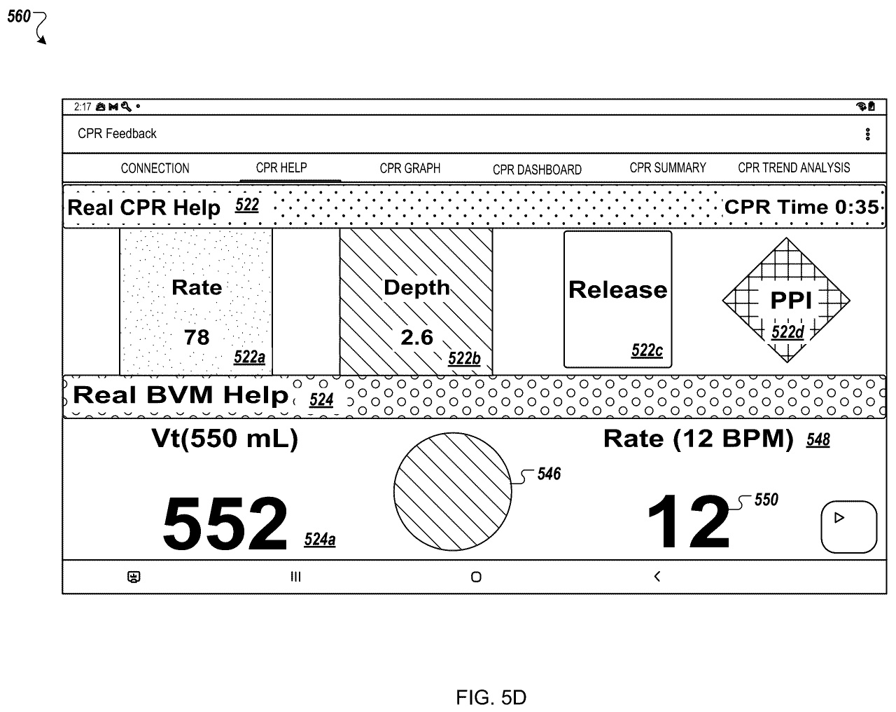

Beneath the compression graph 442, the screen shot 460 includes the first ventilation graph segment 452a corresponding to the second rescuer 444a delivering ventilations and the second ventilation graph segment 452b corresponding to the first rescuer 444a delivering ventilations for a time period after the second rescuer 454a. Thus, by reviewing the screen shot 460, it is apparent that the first rescuer 444a switched roles with the second rescuer 444b between the first graph segments 442a, 452a and the second graph segments 442b, 452b. FIG. 5A through FIG. 5D illustrate example screen shots of a portable computing device configured for role-based feedback to a supervisor overseeing rescuers donning wearable computing devices at an medical emergency scene. The screen shots, in some implementations, begin with a log-in screen illustrating a login status 502 for the device having the display as well as a connection status 506 for wearable devices configured to communicate with the portable device presenting the display 500. The connection, in some examples, can include a Bluetooth, Wi-Fi, radio frequency (RF), and/or Zigbee connection.

Turning to the login status 502, in some implementations, the portable device may be connected to a network, such as a Wi-Fi network, for communicating with a remote network or device, such as the network 218 and computing system 220 of FIG. 2. As illustrated, a connect control 504, when selected, may cause the device to attempt to connect to the remote network or device.

The connection status 506, in some implementations, presents information regarding wearable devices connected to the portable device. For example, rescuer names 508 and status 510 are presented. In other embodiments, wearable device identifiers may be included instead of or in addition to rescuer names 508 (e.g., “K. Smith” and “R. Miller”). The status 510 column lists whether each device is connected to the portable computing device or disconnected. In other embodiments, the status may include, in some examples, a role, a time since connecting, a length of time connected, a last active timestamp, and/or an experience level/employee grade/medical training designation associated with each rescuer. In illustration, EMT personnel may be differentiated from nurses.

Turning to FIG. 5B, in some implementations, user interface 520 presents an overview screen including a compression synopsis region 522 and a ventilation synopsis region 524. The user interface 520, for example, may allow the supervisor to view current metrics regarding rescue efforts. The user interface 520, for example, includes similar icons and information as presented in the example wearable device user interfaces of FIG. 3C through FIG. 3G.

In the compression synopsis region 522, in some implementations, a rate icon 522a presents a value as well as a visual indication of sufficiency of rate of compressions, a depth icon 522b presents a value as well as a visual indication of sufficiency of a depth of compressions, a release icon 522c presents a visual indication of sufficiency of release between compressions, and a PPI icon 522d presents a visual indication of sufficiency of both rate and depth of compressions.

In the ventilation synopsis region 524, in some implementations, a tidal volume 524a is presented to review the volume of the most recent ventilation, a volume icon 524c is presented to visual indicate the relative sufficiency of the most recent ventilation, a countdown bar 524b provides a visual indication of percentage of time to next ventilation as well as a numeric indication of number of seconds (e.g., “7”), and a rate value 524d indicates a ratio of compressions to ventilations (e.g., “30:2”).

FIG. 5C and FIG. 5D illustrate alternative design layouts to the user interface 520 of FIG. 5B. In the user interface 540 of FIG. 5C, as illustrated, a rate indicator 542 and a depth indicator 544 each include a numeric value, where the numeric value itself is adjusted in color and/or fill type to indicate relative sufficiency of the value. Further, in the ventilation region 524, a sufficiency of ventilation icon 546 provides a visual indication of sufficiency of tidal volume of the ventilation, while a numeric value 548 in the ventilation icon 546 provides an indication of number of seconds until the next ventilation.

Turning to FIG. 5D, the sufficiency of ventilation icon 546 lacks a numeric value providing an indication of number of seconds until the next ventilation. Further, a rate of ventilation 548 indicates that the rate is 12 breaths per minute (BPM), and a numeric visual indicator 550 illustrates a present BPM value (e.g., “12”). The numeric visual indicator 550 may further include a visual indication of sufficiency, for example in a color of the numeric value.

Returning to the method 620, while rescue efforts are ongoing, in some implementations, the method 620 continues to repeat by returning to waiting to receive another data transmission with an activity value (622).

Although described as a particular series of operations, in some embodiments, more or fewer steps may be included in the method 620. For example, in some embodiments, if one or more stored activity values have a timestamp earlier than the past T amount of time, rather than discarding the oldest activity value (630), all values having a timestamp of current-T or older may be discarded. Similarly, in some embodiments, prior to calculating the combined node activity value (636), it may be confirmed that no more than T time has passed since the timestamp of any of the activity values. In further embodiments, certain steps of the method 620 may be performed in a different order or in parallel. For example, although described as a linear operation for simplicity, activity values may be obtained periodically and/or in near real time with continuous updating and comparison of the node activity values. Other modifications of the method 620 are possible while remaining within the scope and extent of the method 620.

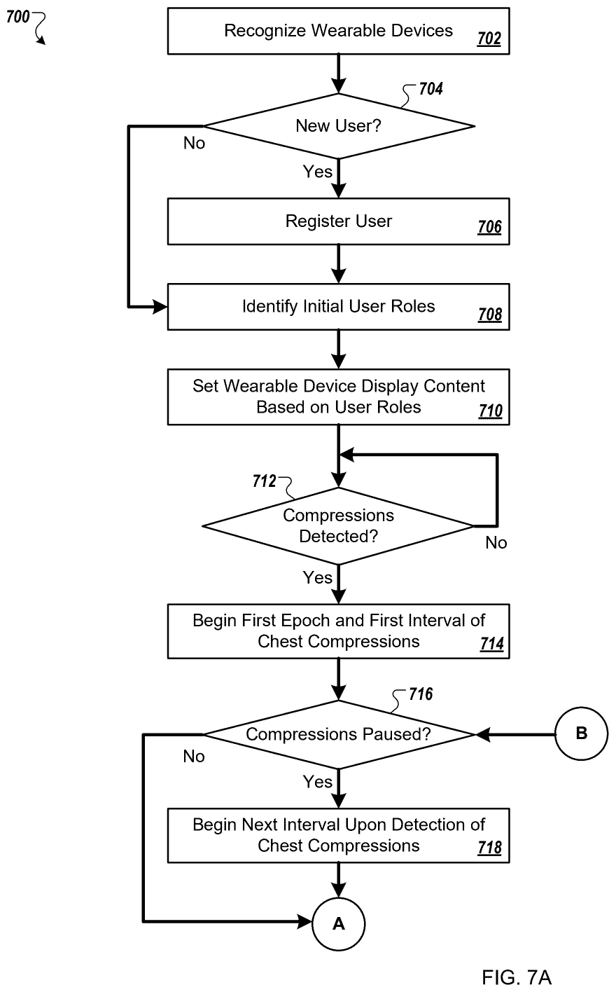

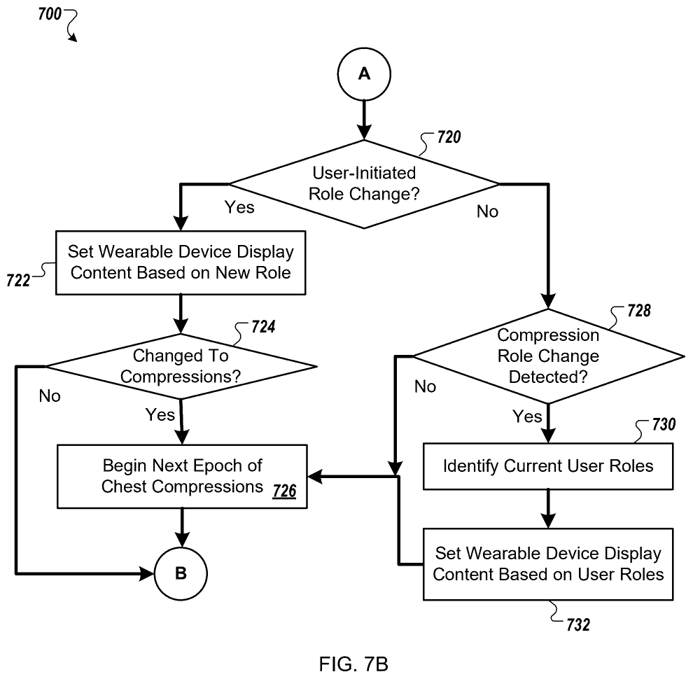

FIG. 7A and FIG. 7B illustrate a flow chart of an example method 700 for tracking role changes of a team of rescuers throughout a rescue at an emergency medical scene. The method 700, in some examples, may be performed by the defibrillation/monitor device 108 of FIG. 1A through FIG. 1C, the portable computing device 114 of FIG. 1A, the defibrillator/monitor 212 of FIG. 2, or the computing device 216 of FIG. 2.

Turning to FIG. 7A, the method 700, in some implementations, begins with recognizing wearable devices (702). The wearable devices, in some examples, may be detected via a wireless transmission such as a near field communication connection, Bluetooth, Wi-Fi, radio frequency signal, or other wireless communication protocol. The wireless transmission may require close proximity, such as low-power Bluetooth or near-field communication (NFC), image-based QR-codes, or radio frequency (RF) identification, including a tap or bump between the wearable device and the computing device performing the method 700. In some embodiments, recognition includes a location/proximity recognition (e.g., enabled by the wireless or image-based techniques mentioned above). In some embodiments, recognition includes one or more of a switch actuation, pressing a button, a gestural code, a sound/vibration, or a voice command/recognition. For example, to initiate pairing with the computing device performing the method 700, the user of the wearable device may activate a pairing protocol through a virtual or physical control mechanism. Additional authentication options are described in U.S. Pat. No. 10,888,229 entitled “Establishing Secure Communication at an Emergency Care Scene” and issued Jan. 12, 2021, the entirety of contents of which is hereby incorporated by reference. Recognizing the wearable device may include receiving identification information regarding the wearable device such as, in some examples, an identifier of the wearable device, an identifier of a rescuer registered with the wearable device, and/or a settings profile of the wearable device (e.g., default role, presentation preferences, etc.).

In some embodiments, a wearable device is recognized in part through initiation of a pairing process via a user interface of the wearable device. For example, as described in relation to FIG. 3A, the pairing process may be initiated via the settings icon 312. In another example, as described in relation to FIG. 3B, the pairing process may be initiated via a machine-readable code icon 316.

In some implementations, if the wearable device is recognized as a new user (704), the user is registered (706). For example, if the wearable device is being paired with the computing device performing the method 700 for the first time and/or with the emergency medical system generally for the first time, the device may be registered as a new device or new “user” of the computing device and/or the emergency medical system. Registering, for example, may include storing identification information of the wearable device. For example, a wearable device identifier may be associated with a rescuer identification.

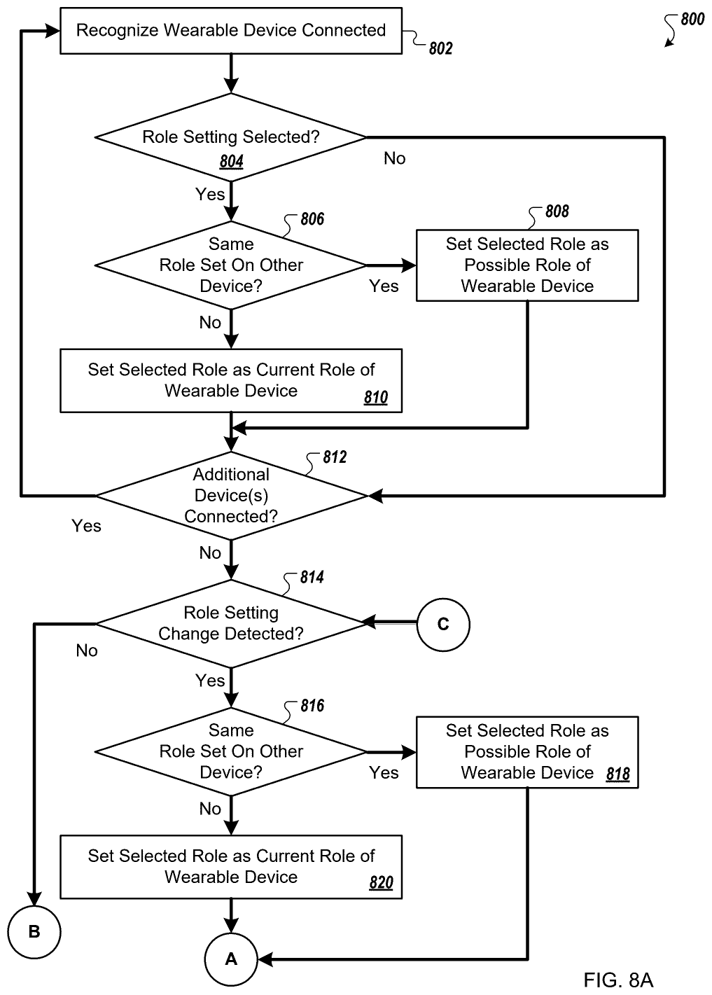

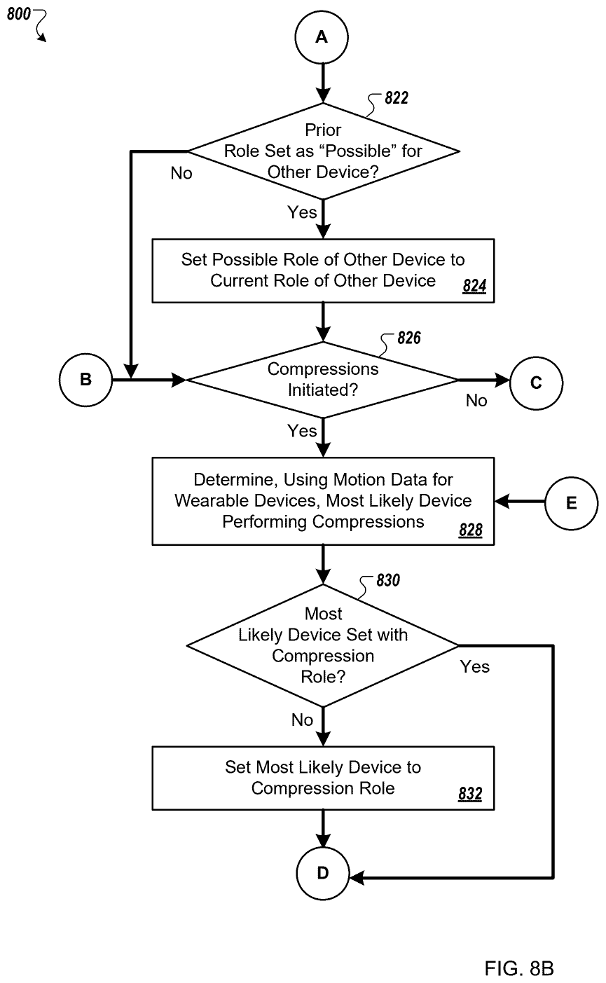

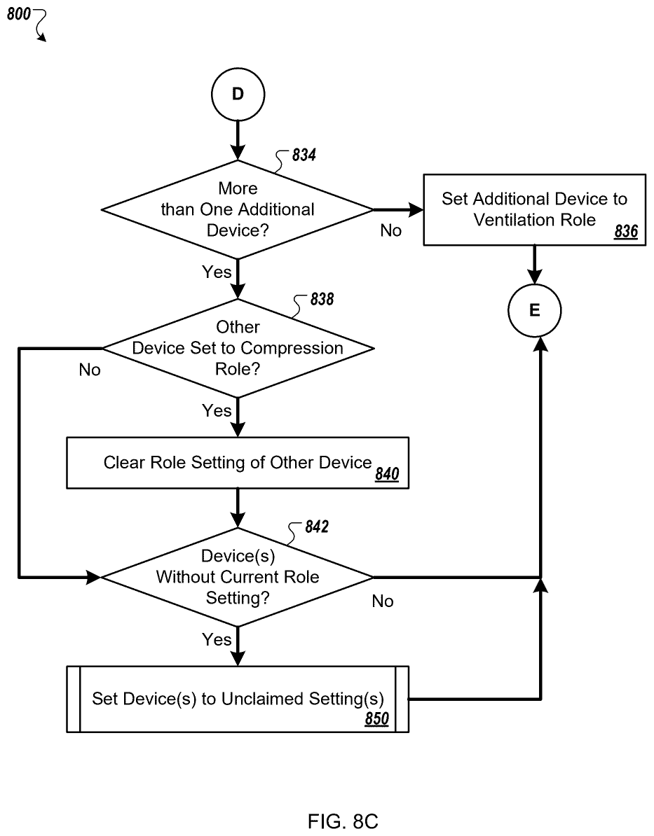

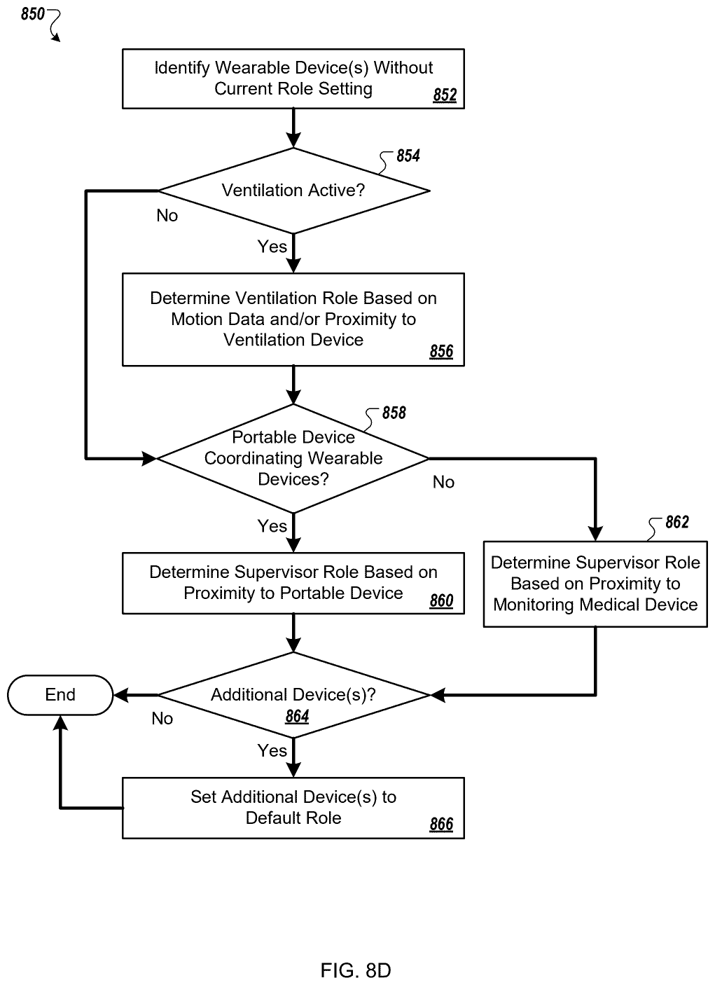

In some implementations, initial user roles are identified (708). The initial roles, in some implementations, are identified using a portion of a method 800 of FIG. 8A through FIG. 8D, described below. The initial roles, in some examples, can include a compression delivery role, a ventilation delivery role, and a supervisor role. In some embodiments, one or more rescuers may select an initial role via a user interface of the wearable device, for example as described in relation to FIG. 3A.

In some implementations, the wearable device display content for each wearable device is set based on the initial user roles (710). The display content, for example, may be presented in one of the forms described in relation to FIG. 3C through FIG. 3I. The setting of the display mode, for example, may be triggered by a signal transmitted from the computing device performing the method 700 to each wearable device.

If compressions are detected (712), in some implementations, a first epoch and a first interval of chest compressions are begun (714). Each epoch, for example, may include one or more rounds of compressions followed by ventilations during which time a same rescuer performs compressions. The epochs, for example, may be tracked for graphing purposes and/or metric generation purposes (e.g., an average length of time a same rescuer spends performing compressions, an average length of time of performing compressions per each rescuer at a rescue scene prior to taking a break, etc.). The epoch may be included, for example, in a CPR tracking log and/or as metadata to a data file collecting compression metrics. Further, identification of a corresponding rescuer performing at least the compression role may be added to the tracking log and/or metadata portion of the data file.

In some implementations, if compressions are paused (716) a next compression interval is begun upon detection of further chest compressions (718). Each compression interval, for example, may involve a time period of compression delivery followed by a time period of ventilation delivery. Further, some intervals may involve intervention therapy in addition to or instead of ventilation delivery, such as delivering a drug or to providing defibrillation therapy. Intervals, for example, may be tracked for graphing purposes and/or metric generation purposes.

Turning to FIG. 7B, in some implementations, if a user of one of the wearable devices initiates a role change (720), the wearable device display content of the user's wearable device is set based on the new role (722). Role change initiation, for example, may be performed via the user interface as described in relation to FIG. 3A. In some embodiments, upon role change, the wearable device is provided with different data, such as the compression data 136a rather than the ventilation data 136b, as described in relation to FIG. 1A. In other embodiments, upon role change, the wearable device is configured to present information using a different portion of the data being supplied to the wearable device, where both ventilation and compression data is always supplied to the wearable device.

If the new role is the compressions role (724), in some implementations, the next epoch of chest compressions is begun (726), and the method 700 returns to detecting whether compressions have paused (716).

If, instead, a user does not initiate a role change (720) but instead a role change of the user performing compressions is detected (728), in some implementations, current user roles are identified (732). For example, in some instances, if the rescuer previously performing ventilations is now performing chest compressions, then another rescuer must be performing ventilations. Or, for roles other than performing chest compressions where the activity value determination is not used, then user input may be employed to confirm the particular role of the user for a corresponding period of time. The role changes may be detected, for example, using at least a portion of the method 800 of FIG. 8A through 8D.

In some implementations, the wearable device display content is set based on the current user roles (732). The display content, for example, may be set as described in relation to operation 722.

In some implementations, the next epoch of chest compressions is begun (726), and the method 700 returns to detected if compressions have paused (716).

Although described as a particular series of operations, in some embodiments, more or fewer steps may be included in the method 700. For example, in some embodiments, a user of the computing device performing the method 700, such as the portable device 114 of FIG. 1A, be configured to initiate a role change (720) on behalf of a wearable device. In further embodiments, certain steps of the method 700 may be performed in a different order or in parallel. For example, identifying initial user roles (708) may be iteratively performed while recognizing wearable devices (702). Other modifications of the method 700 are possible while remaining within the scope and extent of the method 700.

FIG. 8A through 8D illustrate a flow chart of an example method 800 for identifying roles of each rescuer in a team of rescuers at an emergency medical scene. The method 800, in some examples, may be performed by the defibrillation/monitor device 108 of FIG. 1A through FIG. 1C, the portable computing device 114 of FIG. 1A, the defibrillator/monitor 212 of FIG. 2, or the computing device 216 of FIG. 2.

In some implementations, the method 800 begins with recognizing a wearable device is connected to an emergency medical system (802). The wearable device may be recognized, for example, as described in relation to operation 702 of FIG. 7.

In some implementations, if a role setting is detected (804), it is determined whether the same role is set on another device connected to the emergency medical system (806). For example, multiple devices may default to a same setting or be initiated to a same setting by different rescuers at the emergency scene.

If the same role is set on another wearable device (806), in some implementations, the selected role is set as a possible role of the wearable device (808). For example, role settings may be accepted on a “first identified” basis such that, due to the conflict between the two devices, the first to register for the role may be assigned the role while the other is flagged as potentially belonging to the role. In other implementations, the later user-set role may supersede the earlier user-set role (e.g., the selected role may be set as the role, and on the other device the role may be modified to possible role).

If, instead, the same role is not set on another wearable device (806), in some implementations, the selected role is set as the current role of the wearable device (810). Setting the role, for example, may include correlating the identifier of the wearable device with an identifier representing the role and/or activating role-related feedback on the corresponding wearable device.

In some implementations, as additional wearable devices connect (812), the wearable devices are recognized (802) and roles are determined as described in relation to operations 804 through 810.

In some implementations, once the wearable devices are all connected (812), if a role setting change is detected (814), it is determined if the same role is set on another device (816). If the role is already set on another device (816), in some implementations, the selected role is set as a possible role of the wearable device (818). Similar to operations 804 and 806, after multiple devices have been recognized (e.g., after a rescue operation has been initiated), rescuers may alter settings of devices.

If, instead, the role is not already set on another device (816), in some implementations, the selected role is set as the current role of the wearable device (820). Setting the role, for example, may be performed as described in relation to operation 810.

Turning to FIG. 8B, in some implementations, if the prior role of the device was set to possible on another device (822), then the possible role setting of the other device is updated to be the current role of the other device (824). In this manner, if a conflict no longer exists between multiple wearable devices, a later device to set the role (e.g., the “possible” as discussed in relation to operation 818) is now set as having that role.

In some implementations, if compressions have not yet been initiated (826), the method 620 returns to detecting role changes (814). Compression initiation, for example, may be identified based on feedback received from medical equipment, such as the defibrillation/monitor device 108 of FIG. 1A.

If, instead, compressions have been initiated (826), in some implementations, the motion data for the wearable devices is used to determine the most likely device worn by the rescuer performing compressions (828). For example, the method 620 of FIG. 6B-1 and FIG. 6B-2 may be executed to determine the most likely device worn by the rescuer performing the compressions role.

In some implementations, if device determined to be the most likely to be worn by the rescuer performing compressions is not currently set with the compression role (830), the role setting of the most likely device is set to the compression role (832). Role setting, for example, may be performed as described in relation to operation 810.

Turning to FIG. 8C, in some implementations, if there is only one additional device (834), the other wearable device is set to the ventilation role (836) and the method 800 returns to using motion data to determine the most likely device worn by the rescuer performing compressions (828). For example, the method 800 may continuously or periodically perform calculations (e.g., as activity values are received and/or as milestones are achieved in therapy (e.g., X amount of time after each ventilation, after any pause in compressions, etc.) for determining the most likely device worn by the rescuer performing compressions.

In some implementations, if three or more wearable devices are involved in the method 800, if another wearable device is set to the compression role (838), the compression role setting is cleared on the other wearable device (840). For example, the previous rescuer performing compressions has likely switched to a different role, so that role will need to be determined.

If there are one or more devices lacking a current role setting (842), in some implementations, the device(s) without a current role setting are set to any unclaimed settings (see method 850). Roles, for example, may be determined in a hierarchical order and/or an order based on the type of equipment deployed and/or emergency scene.

Turning to FIG. 8D, a method 850 is provided for matching wearable devices with unclaimed role settings. In some implementations, the method 850 begins with identifying the wearable device(s) without a current role setting (852). A table of matches between identifiers and corresponding roles, for example, may be accessed to determine which identifiers lack a role setting (or, conversely, include a “possible” role setting as described in relation to method 800).

In some implementations, if ventilation delivery is active (854), the ventilation role is determined based on motion data and/or proximity to the ventilation device (856). A short-range or other wireless protocol, for example, may be used to discern proximity.

In some implementations, if a portable device is coordinating role settings and data distribution among the wearable devices (858), a supervisor role is determined based on proximity of a wearable device to the portable computing device (860). A short-range or other wireless protocol, for example, may be used to discern proximity. Conversely, a biometric signature or log-in to the portable device may be used to set the initial supervisor role to a particular rescuer identifier.

If a medical device is coordinating role settings and data distribution among the wearable devices (858), in some implementations, a supervisor role is determined based on proximity to the medical device monitoring the wearable devices (862). A short-range or other wireless protocol, for example, may be used to discern proximity.

In some implementations, if one or more additional wearable devices have been detected (864), the additional device(s) are set to default roles (866). Additional roles, in some examples, may include a medication dosage/delivery timing role or a first aid/medical triage role. The wearable devices, for the additional roles, may provide context-sensitive guidance (e.g., decision support), a listing of protocol steps, or other prompting. Further, in some embodiments, the rescuer executing one of the additional roles may interact with the wearable device to confirm execution (e.g., to confirm drug delivery, etc.). In an illustrative example, a gesture such as vertical shaking for yes, horizontal shaking for no, may be used to indicate completion of a step and/or to respond to yes/no prompts within context-sensitive guidance. In another example, victim vital measurements or metrics may be presented in the wearable device display for at least a portion of the additional roles. In some embodiments, a supervisor (e.g., using a portable computing device such as the portable device 114 of FIG. 1A and FIG. 1B or the portable device 216 of FIG. 2) may assign the additional roles via a user interface displayed on the portable computing device.

Although described as a particular series of operations, in some embodiments, more or fewer steps may be included in the method 850. For example, an employee seniority and/or title associated with the identifier may be determined and used in assigning roles. The highest-ranking rescuer at the scene, in an illustrative example, may be presumed as initially performing the role of supervisor. In further embodiments, certain steps of the method 850 may be performed in a different order or in parallel. For example, the supervisor role may be determined prior to the ventilation role, for example based on proximity. Other modifications of the method 850 are possible while remaining within the scope and extent of the method 850.

FIG. 9A is a screen shot of an example performance report 900 presenting chest compression-related metrics corresponding to multiple rescuers who performed the chest compression role at an emergency medical scene, such as a first rescuer 902a and a second rescuer 902b. The performance report 900, for example, may be prepared by the remote computing system 220 for presentation at the user interface 240.

In some implementations, for each rescuer 902a, 902b, a pie graph 904a, 904b is illustrated, visually representing a percentage of time compressions were actively being delivered in comparison to the time at which no compressions were being delivered. This corresponds, for example, to a CPR pauses bar graph 906a, 906b illustrating the relative time periods at which pauses between compressions were under 5 seconds, between 5 and 10 seconds, or over 10 seconds. For example, the first rescuer 902a was performing active compressions 69.21% of the time, and 48.48% of the pauses between compressions under five seconds, while the second rescuer 902b was performing active compressions 59.21% of the time, and 58.82% of the pauses were under five seconds. However, rescuer 1 902a only had pauses over 10 seconds 38.53% of the time, while rescuer 2 902b had pauses over 10 seconds nearly a quarter of the time (23.53%).

Further, in some implementations for each rescuer 902a, 902b, a manual depth variability bar chart 908a, 908b presents relative periods of time where depth is in target, too deep, and too shallow. Further, a manual rate variability bar chart 910 visually represents a percentage of time the compression rate was in range in comparison to too fast and too slow.

The bar charts 906, 908, and 910, in some implementations, represent metrics for one epoch of potentially multiple epochs during which either of the rescuer 902a, 902b performed chest compressions. For example, previous controls 910a, 910b and next controls 912a, 910b, when actuated, may move the viewer to a different epoch. Further, the pie graphs 904a,b may represent an overall statistical analysis encompassing all epochs.

In other embodiments, additional metrics may be presented such as, in some examples, compression release velocity and/or compression pause length. The compression release velocity and/or the compression pause length may be identified, for example, as “in target” (e.g., percentage time in target range) versus “out of target”, such as a percentage of time compression velocity was within target. In another example, the compression release velocity and/or the compression pause length may be identified as a numeric value or based on a numeric value, such as an average compression velocity over a set of compressions or over the time a particular rescuer performed compressions. In a further example, one or more metrics representing PPI indicator trend(s) (e.g., sufficiency of both depth and velocity) may be illustrated.

FIG. 9B is a screen shot of example ventilation performance report 920 presenting ventilation-related metrics corresponding to multiple rescuers who performed the ventilation role at an emergency medical scene, such as a first rescuer 922a and a second rescuer 922b. The performance report 920, for example, may be prepared by the remote computing system 220 for presentation at the user interface 240.

In some implementations, for each rescuer 922a, 922b, a ventilation volume bar graph 926a, 926b illustrates, over a time period 924a, 924b, relative sufficiency of ventilations delivered. The ventilation volume bar graph 926a, 926b, for example, illustrates a percentage of deliveries for which the volume was insufficient (e.g., under 300 mL), sufficient (e.g., within a target range of 300 to 600 mL), and exceeding target range (e.g., >600 mL).

Similarly, in some implementations, a ventilation rate bar graph 928a, 928b illustrates, for each rescuer 922a, 922b, relative sufficiency of ventilation delivery rate over the time period 924a, 924b. The ventilation rate bar graph 928a, 928b, for example, illustrates a percentage of deliveries for which the delivery was early (e.g., less than eight minutes), within a target delivery time (e.g., between eight and fifteen minutes, or late (e.g., over fifteen minutes between ventilation sessions).

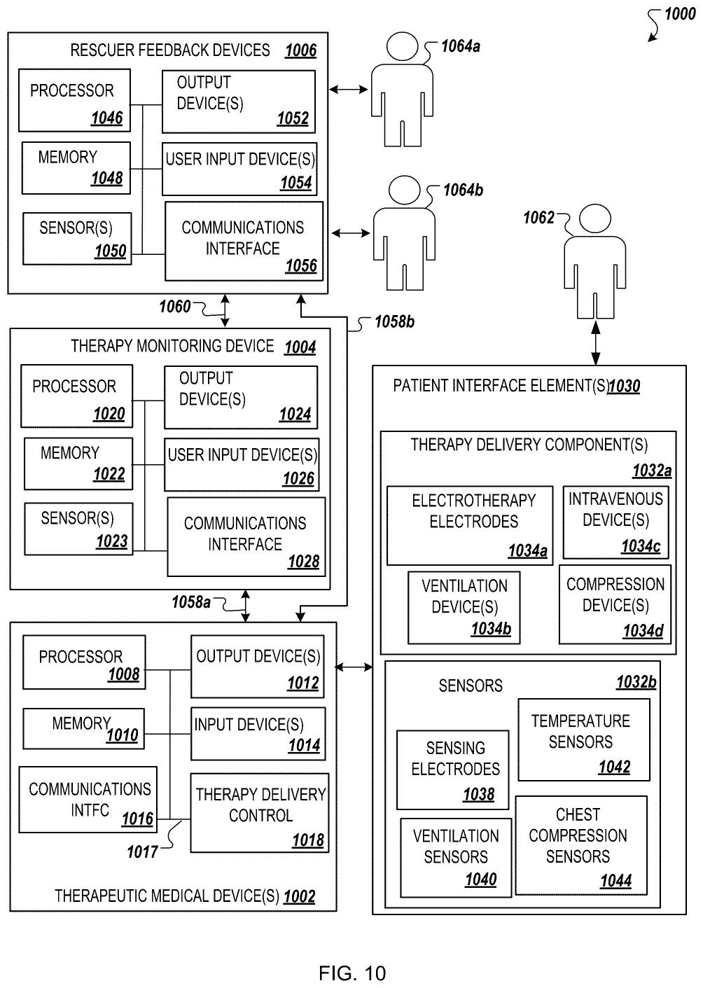

The bar charts 926 and 928, in some implementations, represent metrics for one epoch of potentially multiple epochs during which either of the rescuer 922a, 922b performed chest compressions. For example, previous controls 930a, 930b and next controls 932a, 932b, when actuated, may move the viewer to a different epoch. FIG. 10 is a block diagram of an example system 1000 of computing devices configured for use at an emergency medical scene. The devices include a therapy monitoring device 1004, one or more therapeutic medical devices 1002, and rescuer feedback devices 1006 for providing feedback to a team of rescuers 1064. The therapeutic medical devices 1002 may interface with a patient 1062 via a set of patient interface elements 1030. The devices 1002, 1004, and 1006 of the system 1000, for example, may be deployed at an emergency medical scene, such as the scene 100 of FIG. 1A, the scene 150 of FIG. 1B, the scene 170 of FIG. 1C, or the scene 200 of FIG. 2. The devices 1002, 1004, and/or 1006 may represent various devices deployed at the emergency medical scenes 100, 150, and/or 200.

For example, the medical therapy may be electrical therapy (e.g., defibrillation, cardiac pacing, synchronized cardioversion, diaphragmatic or phrenic nerve stimulation), and the medical device 1002 may be a defibrillator, a defibrillator/monitor, a mechanical ventilator such as the ZOLL Z-Vent, and/or another medical device configured to provide electrotherapy. As another example, the medical therapy may be chest compression therapy for treatment of cardiac arrest and the medical device 1002 may be a mechanical chest compression device such as a belt-based chest compression device or a piston-based chest compression device. In other examples, the medical therapy may be ventilation therapy, therapeutic cooling or other temperature management, invasive hemodynamic support therapy (e.g., Extracorporeal Membrane Oxygenation (ECMO)), etc. and the medical device 1002 may be a device configured to provide a respective therapy. Further, in some embodiments, the medical device 1002 includes a combination of one or more of these examples. The therapeutic medical device 1002 may include patient monitoring capabilities via one or more sensors 1032b. In a particular example, the medical device may be the defibrillation/monitor device 108 of FIG. 1A through FIG. 1C or the defibrillation device 212 of FIG. 2.

Turning to the therapeutic medical devices 1002, each medical device 1002 may include a processor 1008, a memory 1010, a communications interface 1016, one or more input devices 1014, one or more output devices 1012, and a therapy delivery control module 1018, all interconnected by a communications bus 1017. The therapy delivery control module 1018 enables delivering of a medical therapy.

In some embodiments, the therapeutic medical device 1002 is an integrated therapy delivery/monitoring device within a single housing. The single housing may surround, at least in part, at least a portion of the patient interface elements 1030, including, for example, some therapy delivery components 1032a and monitoring components such as sensors 1032b. In other embodiments, the medical device 1002 is a modular therapy delivery/monitoring device, with patient therapy components 1032a in one unit communicatively coupled to a patient monitoring unit with monitoring components (e.g., sensors 1032b) but without therapy delivery components 1032a.

The patient interface elements(s) 1030 include one or more therapy delivery component(s) 1032a and/or one or more sensor device(s) 1032b. The therapy delivery component(s) 1032a are configured to deliver therapy to the patient 1062 and may be configured to couple to the patient 1062. For example, the therapy delivery component(s) 1032a may include one or more of electrotherapy electrodes including defibrillation electrodes and/or pacing electrodes, chest compression devices (e.g., one or more belts or a piston), ventilation devices (e.g., a mask and/or tubes), drug delivery devices, etc. The medical device 1002 may include the one or more therapy delivery component(s) 1032a and/or may be configured to couple to the one or more therapy delivery component(s) 1032a in order to provide medical therapy to the patient 1062. The therapy delivery component(s) 1032a may be configured to couple to the patient 1062. For example, one of the rescuers 1064 may attach the electrodes to the patient 1062 and the medical device 1002 (e.g., a defibrillator or defibrillator/patient monitor) may provide electrotherapy to the patient 1062 via the defibrillation electrodes. In some embodiments, the medical device 1002 couples to at least a portion of the patient interface elements(s) 1030 via a wired or wireless communications protocol supported by the communications interface 1016.

The medical device(s) 1002, in some implementations, include, incorporate, and/or are configured to couple to the one or more sensor(s) 1032b. The sensor(s) 1032b may be configured to provide signals indicative of sensor data to the medical device 1002. The sensor(s) 1032 may be configured to couple to the patient 1062. In some embodiments, the medical device 1002 couples to at least a portion of the sensors 1032b via a wired or wireless communications protocol supported by the communications interface 1016.

In some embodiments, the sensor(s) 1032b include sensing electrodes 1038 such as one or more cardiac sensing electrodes, a chest compression sensor 1044, and/or ventilation sensors 1040. The sensing electrodes 1038 may be conductive and/or capacitive electrodes configured to measure changes in a patient's electrophysiology to measure the patient's ECG information. The sensing electrodes 1038 may further measure the transthoracic impedance and/or a heart rate of the patient 1062. The one or more sensors 1032b may generate signals indicative of physiological parameters of the patient 1062. For example, the physiological parameters may include one or more of at least one vital sign, an ECG, blood pressure, heart rate, pulse oxygen level, respiration rate, heart sounds, lung sounds, respiration sounds, tidal CO2, saturation of muscle oxygen (SMO2), arterial oxygen saturation (SpO2), cerebral blood flow, electroencephalogram (EEG) signals, brain oxygen level, tissue pH, tissue fluid levels, physical parameters as determined via ultrasound images, parameters determined via near-infrared reflectance spectroscopy, pneumography, and/or cardiography, etc. The ultrasound images may include ultrasound images of a patient's heart, carotid artery, and/or other components of the cardiovascular system. Additionally or alternatively, the one or more sensors 1032b may generate signals indicative of chest compression parameters, ventilation parameters, drug delivery parameters, fluid delivery parameters, etc.

In some embodiments, the sensor(s) 1032b include ventilation sensors 1040. The ventilation sensor(s) 1040 may include spirometry sensors, flow sensors, pressure sensors, oxygen and/or carbon dioxide sensors such as, for example, one or more of pulse oximetry sensors, oxygenation sensors (e.g., muscle oxygenation/pH), O2 gas sensors and capnography sensors, and combinations thereof.

In some embodiments, the sensor(s) 1032b include temperature sensors 1042. The temperature sensors 1042 may include an infrared thermometer, a contact thermometer, a remote thermometer, a liquid crystal thermometer, a thermocouple, a thermistor, etc. and may measure patient temperature internally and/or externally.

In some embodiments, the sensor(s) 1032b include chest compression sensor(s) 1044. The chest compression sensor(s) 1044 may include one or more motion sensors including, for example, one or more accelerometers, one or more force sensors, one or more magnetic sensors, one or more velocity sensors, one or more displacement sensors, etc. The chest compression sensor(s) 1044 may be incorporated into, in some examples, a compression puck, a smartphone, a hand-held device, a wearable device, etc. The chest compression sensors 1044 may be configured to detect chest motion imparted by one of the rescuers 1064 and/or an automated chest compression device (e.g., a belt system, a piston system, etc.). The chest compression sensors 1044 may provide signals indicative of chest compression data including displacement data, velocity data, release velocity data, acceleration data, compression rate data, dwell time data, hold time data, blood flow data, blood pressure data, etc. In some implementations, the sensing electrodes 1038 and/or the electrotherapy electrodes 1034a may include or be configured to couple to the chest compression sensors 1044.

In addition to delivering therapy to the patient 1062, in some implementations, the therapy delivery component(s) 1032a may include, be coupled to, and/or function as sensors and provide signals indicative of sensor data to the medical device 1002. For example, the defibrillation electrodes may be configured as cardiac sensing electrodes 1038 as well as electrotherapy electrodes 1034a and may provide signals indicative of transthoracic impedance, electrocardiogram (ECG), heart rate and/or other physiological parameters. As another example, a therapeutic cooling device may be an intravenous cooling device. Such a cooling device may include an intravenous (IV) device 1034c as a therapy delivery component 1032a configured to deliver cooling therapy and sense the patient's temperature as a temperature sensor 1042. For example, the IV device may be a catheter that includes saline balloons configured to adjust the patient's temperature via circulation of temperature controlled saline solution. In addition, the catheter may include a temperature probe configured to sense the patient's temperature. As a further example, an IV device 1034c may provide therapy via drug delivery and/or fluid management. The IV device 1034c may also monitor and/or enabling monitoring of a patient via blood sampling and/or venous pressure monitoring (e.g., central venous pressure (CVP) monitoring).

The medical device 1002, in some implementations, is configured to receive sensor signals from the therapy delivery component(s) 1032a and/or the sensor(s) 1032b and to process the sensor signals to determine and collect patient data. The patient data may include patient data which may characterize a status and/or condition of the patient 1062 (e.g., physiological data such as ECG, heart rate, respiration rate, temperature, pulse oximetry, non-invasive hemoglobin parameters, capnography, oxygen saturation (SpO2), end tidal carbon dioxide (EtCO2), invasive blood pressure (IBP), non-invasive blood pressures (NIBP), tissue pH, tissue oxygenation, Near Infrared Spectroscopy (NIRS) measurements, etc.). Additionally or alternatively, the patient data may characterize the delivery of therapy (e.g., chest compression data such as compression depth, compression rate, etc.) and/or the patient data may characterize a status and/or condition of the medical equipment used to treat the patient (e.g., device data such as shock time, shock duration, attachment of electrodes, power-on, etc.). The patient data may be collected by the processor 1008 via the communications interface 1016 and stored to the memory 1010.

In some implementations, the therapy delivery control module 1018 is configured to couple to and control the therapy delivery component(s) 1032a. In one example, the therapy delivery control module 1018 of the medical device 1002 may be an electrotherapy delivery circuit that includes one or more capacitors configured to store electrical energy for a pacing pulse or a defibrillating pulse. The electrotherapy delivery circuit may further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 1018 may be a compression device electro-mechanical controller configured to control a mechanical compression device. As a further example, the therapy delivery control module 1018 may be an electro-mechanical controller configured to control drug delivery, temperature management, ventilation, and/or other type of therapy delivery.

A user, in some implementations, initiates therapy and/or adjusts therapy parameters via one or more input devices 1014 of the medical device 1002. The input devices 1014, in some examples, may include one or more virtual or physical controls, such as buttons, switches, dials, and/or touch controls presented at a touch screen display of the medical device 1002. Further, the input devices 1014 may include a communications interface 1016 for receiving instructions from a remotely located control device and/or remote application such as a smart phone application.

In some implementations, a user receives feedback from the medical device 1002 and/or reviews a portion of the patient data via one or more output devices 1012. The output devices 1012, in some examples, may include a display screen for presenting a portion of the patient data and/or metrics derived therefrom (e.g., as calculated by the processor 1008). The output devices 1012 may include a speaker for generating alarms, audible instructions, or pacing tones (e.g., for prompting timing of chest compressions, ventilations, etc.).

Although shown as separate elements, one or more of the elements of the medical device 1002 may be combined into one or more discrete components and/or may be part of the processor 1008. The processor 1008 and the memory 1010 may include and/or be coupled to associated circuitry to perform the functions described herein.

In some implementations, the medical device 1002 is configured to communicate with the therapy monitoring device 1004 and/or the set of rescuer feedback devices 1006 via the communications interface 1016. The communications interface 1016 may be configured to communicate with the therapy monitoring device 1004 via wired and/or wireless communicative couplings. Further, the communications interface 1016 may be configured to communicate with the rescuer feedback devices 1006 via wireless communicative couplings. The communications interface 1016 may be configured to communicate using one or more communications protocols. In some examples, the communications interface 1016 may be configured to communicate via a wireless coupling such as a cellular network including FirstNet by First Responder Network Authority, EDGE, 3G, 4G, and 5G wireless cellular systems. The wireless network can also include Wi-Fi®, Bluetooth®, Zigbee®, or another wireless form of communication.

In some implementations, the medical device 1002 receives activity values from the rescuer feedback devices 1006.

The processor 1008 of the medical device 1002, in some implementations, is configured to provide information regarding roles of the rescuers 1064 to the therapy monitoring device 1004. For example, the information regarding roles of the rescuers may be included as a portion of the patient data generated by the medical device 1002. In some embodiments, the processor 1008 is configured to perform the method 620 of FIG. 6B-1 and FIG. 6B-2 to identify roles of the rescuers 1064.

In some implementations, the medical device 1002 provides the patient data to the therapy monitoring device 1004 over a communications coupling 1058a. For example, a communications interface 1028 of the therapy monitoring device 1004 may establish with communications coupling 1058a with the communications interface 1016 of the medical device 1002. The processor 1008 of the medical device 1002 may generate the patient data and provide the patient data to the therapy monitoring device 1004, via the communications interface 1016, in real-time or near real-time to support oversight of the therapy at the therapy monitoring device 1004.

In some implementations, the therapy monitoring device 1004 stores the patient data to a memory 1022. A processor 1020 of the therapy monitoring device 1004 may generate one or more metrics from the patient data and/or apply one or more rules to the patient data for identifying problems with the therapy (e.g., delivery problem, equipment problem, etc.). The processor 1020 may present the patient data, metrics, and/or alarms related to problems via one or more output devices 1024. For example, the therapy monitoring device 1004 may display the user interface 400 of FIG. 4A or the user interface 420 of FIG. 4B on a display screen) for real-time review by a medical professional or supervisor of the rescuers 1064. A user may interact with the therapy monitoring device 1004 to review various aspects of the patient data using one or more user input devices 1026. The user input devices, in some examples, can include buttons, a keyboard, a touch screen, a voice recognition microphone, or a gesture recognition sensor.

The therapy monitoring device 1004, in some embodiments, is a portable computing device such as, in some examples, a laptop computer, tablet computer, mobile phone, or other handheld computing device. The therapy monitoring device 1004, in particular examples, may be the supervisor device 114 of FIG. 1A or the computing device 216 of FIG. 2.

Turning to the rescuer feedback devices 1006, in some implementations, a personal feedback device is allocated to each rescuer 1064 at an emergency scene. The rescuer feedback devices 1006, for example, may include the wearable devices 110a, 110b of FIG. 1A through FIG. 1C and/or the wearable devices 230a, 230b of FIG. 2. As illustrated, each feedback device may include a processor 1046, a memory 1048, one or more sensors 1050, one or more output devices 1052, one or more input devices 1054, and at least one communications interface 1056 for communicating with the therapy monitoring device 1004 and/or the therapeutic medical device(s) 1002.

The communications interface 1056, for example, may receive feedback data from the therapy monitoring device 1004 and/or the therapeutic medical device(s) 1002, and the processor 1046 may prepare the information for presentation via the output device(s) 1052, such as a display interface. The feedback provided by the rescuer feedback devices 1006, in some examples, can be presented in one or more of the formats described in relation to FIG. 3A through FIG. 3I.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A system for identifying role changes among a set of rescuers, the system comprising:
a set of wearable devices configured to be donned by a rescuer, each wearable device of the set of wearable devices comprising
a display,
processing circuitry,
a wireless communication module,
at least one sensor configured to detect motion, and
a non-volatile storage medium,
wherein the processing circuitry is configured via hardware logic and/or configured to execute software logic to perform a plurality of operations, the operations comprising
monitoring a plurality of signals of the at least one sensor,
collecting a set of motion data samples over a predetermined sample time period,
using the set of motion data samples, calculating an activity value representing the predetermined sample time period, and
transmitting, via the wireless communication module, the activity value; and
a computing device comprising
processing circuitry,
a wireless communication module, and
a non-volatile storage medium,
wherein the processing circuitry is configured via hardware logic and/or configured to execute software logic to perform a plurality of operations, the operations comprising
receiving, from each respective wearable device of the set of wearable devices, a plurality of activity values, each activity value representing motions of a respective rescuer of the set of rescuers wearing the respective wearable device during a predetermined time period,
storing, for each wearable device of the set of wearable devices in a storage region of the non-volatile storage medium, a time progression of activity values of the plurality of activity values received from the respective wearable device, and
determining, in near real-time using the time progression of activity values for each device of the set of wearable devices, a given wearable device of the set of wearable devices worn by a determined rescuer of the set of rescuers actively performing chest compressions.

2. The system of claim 1, wherein the computing device is a portable computing device or a medical device.

3. (canceled)

4. The system of claim 1, wherein the processing circuitry of the set of wearable devices is configured to perform operations comprising:
detecting, from the plurality of signals, threshold motion of the respective wearable device;
wherein each motion data sample of the set of motion data samples is collected based at least in part on the detecting.

5. The system of claim 1, wherein the plurality of signals represent at least one of acceleration of the wearable device, a gyroscopic progression of measurements sensed by the wearable device, or one or more magnetic field measurements sensed by the wearable device.

6. The system of claim 1, wherein the determining comprises, for each wearable device of the set of wearable devices:
calculating a combined activity value from the time progression of activity values corresponding to the respective wearable device; and
comparing the combined activity values of each of at least two wearable devices of the set of wearable devices to identify a greatest combined activity value.

7. The system of claim 6, wherein calculating the combined activity value comprises at least one of i) calculating a root mean square (RMS) value, ii) identifying a dominant frequency of compression rate, or iii) applying pattern matching to the time progression of values.

8.-9. (canceled)

10. The system of claim 6, wherein calculating the combined activity value comprises calculating the combined activity value across multiple directions of motion.

11. The system of claim 6, wherein determining the given wearable device most likely worn by the determined rescuer actively performing chest compressions comprises storing, in a second non-volatile storage region, the combined activity value.

12. The system of claim 1, wherein the processing circuitry of the computing device is configured to perform operations comprising, prior to determining the given wearable device, applying noise suppression to the time progression of values.

13. The system of claim 1, wherein the wireless communication module of each wearable device of the set of wearable devices is configured to communicate over a short-range communication protocol.

14. (canceled)

15. The system of claim 1, wherein the processing circuitry of the computing device is configured to perform operations comprising, responsive to determining the given wearable device is most likely worn by the determined rescuer actively performing chest compressions, compression data related to a respective depth of active compression to the processing circuitry of the given wearable device.

16. The system of claim 15, wherein the processing circuitry of the computing device is configured to perform operations comprising formatting at least a portion of the compression data for presentation on the display of the given wearable device.

17. The system of claim 16, wherein formatting the portion of the compression data comprises presenting a current compression rate on the display of the given wearable device.

18. The system of claim 16, wherein formatting the portion of the compression data comprises presenting one of a set of color codes indicative of at least one of an acceptable compression rate, an unacceptable compression rate, an acceptable compression depth or an unacceptable compression depth.

19. The system of claim 16, wherein formatting the portion of the compression data comprises presenting a current compression depth on the display of the wearable device.

20. (canceled)

21. The system of claim 16, wherein formatting the portion of the compression data comprises presenting an indication of sufficiency of release from compression depth.

22. The system of claim 16, wherein the processing circuitry of the computing device is configured to perform operations comprising analyzing at least a portion of the compression data to prompt a timing of a next compression.

23. The system of claim 22, wherein prompting the timing of the next compression comprises providing at least one of an audible signal, a visual signal, or a haptic signal to the determined rescuer via the given wearable device.

24. The system of claim 1, wherein the predetermined sample time period is less than one second.

25. (canceled)

26. The system of claim 1, wherein collecting the set of motion data samples comprises storing each data sample of the set of motion data samples in a first-in-first-out (FIFO) queue.

27. The system of claim 1, wherein a number of the set of motion data samples is at least three.

28. The system of claim 1, wherein the processing circuitry of each wearable device of the set of wearable devices is configured to perform operations comprising:
based on the plurality of signals, detecting threshold motion of the wearable device;
wherein the collecting is responsive at least in part to detecting the threshold motion.

29. The system of claim 1, wherein the processing circuitry of each wearable device of the set of wearable devices is configured to perform operations comprising obtaining, from the computing device, indication of active chest compressions, wherein the determining is responsive at least in part to obtaining the indication of the active chest compressions.

30. The system of claim 1, wherein determining the given wearable device most likely worn by the determined rescuer actively performing chest compressions comprises comparing a timestamp associated with each activity value of the time progression of activity values with a period of time of active chest compressions.

31. (canceled)

32. The system of claim 1, wherein the processing circuitry of the computing device is configured to perform operations comprising setting a role of the determined rescuer wearing the given wearable device to a compression delivery role, wherein a set of roles comprises the compression delivery role and a non-compression delivery role.

33. The system of claim 32, wherein setting the role of the determined rescuer wearing the given wearable device comprises providing, to a patient monitoring device, an identifier associated with the given wearable device.

34. The system of claim 32, wherein setting the role of the determined rescuer comprises switching the compression delivery role from a prior wearable device of the set of wearable devices to the given wearable device.

35. The system of claim 32, wherein:
setting the role of the rescuer comprises beginning a new epoch of a time series of epochs, each epoch corresponding to compression delivery by a same rescuer of the set of rescuers; and
each epoch of the time series of epochs comprises at least one compression interval, wherein each compression interval comprises a compression delivery time period and a ventilation time period.

36. (canceled)

37. The system of claim 1, wherein determining the given wearable device most likely worn by the determined rescuer actively performing chest compressions comprises confirming that each time progression of activity values comprises at least a threshold number of values.

38. The system of claim 37, wherein:
the threshold number of values is at least three; and
storing the time progression of activity values comprises storing up to the threshold number of values.

39.-127. (canceled)
